(19) United States
(12) Patent Application Publication
(10) Pub. No.: US 2026/0227388 A1
(43) Pub. Date: Aug. 6, 2026

(54) FRET-BASED MICROARRAY SCREENING PLATFORMS

(71) Applicant: The General Hospital Corporation, Boston, MA (US)

(72) Inventors: Ralph Mazitschek, Weston, MA (US); Neil Connor Payne, Boston, MA (US)

(21) Appl. No.: 19/150,422

(22) PCT Filed: Jan. 23, 2024
(86) PCT No.: PCT/US2024/012614

§ 371 Date: Jul. 23, 2025

Related U.S. Application Data
(60) Provisional application No. 63/441,176, filed on Jan. 25, 2023.

(51) Int. Cl. G01N 33/542; G01N 21/64; G01N 33/53; G01N 33/68

(52) U.S. Cl. CPC G01N 33/542; G01N 21/6458; G01N 21/6486; G01N 33/5308; G01N 33/6845

(57) ABSTRACT

The present disclosure provides FRET-based microarray platforms, reagents, and methods.

### CROSS-REFERENCE TO RELATED APPLICATIONS

This application is a § 371 U.S. National Phase Application of International Patent Application No. PCT/US2024/012614, filed on Jan. 23, 2024, which claims the benefit of U.S. Provisional Patent Application Ser. No. 63/441,176, filed on Jan. 25, 2023, the entire contents of each of the foregoing are hereby incorporated by reference herein.

### TECHNICAL FIELD

This invention relates to microarray systems that employ a FRET donor/acceptor pairs and are useful for massive and highly accurate analysis of various target molecules, including small molecules and large molecules such as nucleic acids, carbohydrates, and proteins.

### BACKGROUND

The pace of progress in biomedical research directly depends on techniques that allow for reliable, quantitative, sensitive, fast, inexpensive, miniaturized, and massively parallel experimental designs. Microarray-based technologies, including oligonucleotide, carbohydrate, protein, and small-molecule microarrays, are widely used in many areas of biomedical research. All of these microarray approaches follow the same canonical scheme: libraries of the respective target molecules (e.g., DNA, RNA, proteins, peptides, small molecules, carbohydrates, and the like) are immobilized or directly synthesized on a solid substrate (e.g., surface-treated or surface-coated glass or silicon slides) in a defined two-dimensional array. Then the microarray is incubated with a solution containing a fluorescently labeled analyte(s) of interest, allowing the analyte to bind with the target molecule immobilized on the substrate. Following extensive wash steps to eliminate non-specifically retained analyte (e.g., analyte weakly and non-specifically bound to the surface of the substrate itself as opposed to being bound to the target molecule), the microarray is analyzed using a fluorescence array scanner, providing (semi) quantitative information on binding affinities to a given analyte for many target molecules at once. This conventional microarray approach works well for specific and high-affinity interactions, such as oligonucleotide arrays, that are compatible with extensive washing protocols, which are required to eliminate non-specific background signal. But lower affinity interactions, e.g., as typical for small-molecule and other non-oligonucleotide microarrays, are much more difficult to capture with this technique. One problem with the lower affinity interactions is that the extensive washing steps required for the removal of non-specifically bound fluorescent ligands also remove weakly bound specific interactions.

### SUMMARY

The present disclosure is based, at least in part, on a realization that experimental design based on Förster resonance energy transfer (FRET) provides a much-improved microarray platform that offers distinct advantages over existing microarray approaches, including wash-free analysis (experimental protocols avoiding extensive washing of non-specifically retained analyte), reduction of non-specific background signals, reliable detection of analyte/target molecule interactions that are weaker that those typically detectable by conventional microarray systems, and reliable quantification of analyte/target molecule binding affinities and measurement of binding and dissociation kinetics. In one general example, the present disclosure provides a microarray platform where either (i) a substrate surface is functionalized with a suitable fluorophore (a FRET acceptor) in sufficient spatial proximity to surface-immobilized target molecules or (ii) target molecules that are directly functionalized with a fluorophore are immobilized on the surface of the substrate. The substrate is then incubated with a FRET donor (e.g., a CoraFluor)-labeled analyte that has a specific affinity to the target molecule immobilized on the substrate. Only upon binding to the target molecule on the surface, the analyte will be in sufficient special proximity to the donor to allow for the FRET detection process. Importantly, this microarray systems and methods within the instant claims allow, e.g., for wash-free conditions and even enable quantitative thermodynamic and kinetic readouts for detailed studies of target molecule/analyte interactions. Additionally, the location of the FRET donor/acceptor pair can be switched (i.e., substrate surface may be functionalized with a FRET donor, and the ligand analyte may be functionalized with a FRET acceptor).

Unless otherwise defined, all technical and scientific terms used herein have the same meaning as commonly understood by one of ordinary skill in the art to which the present application belongs. Methods and materials are described herein for use in the present application; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the present application will be apparent from the following detailed description and figures, and from the claims.

### DESCRIPTION OF DRAWINGS

FIGS. 1A-1D TR-FRET background. FRET is the non-radiative energy transfer from a donor fluorophore to an acceptor fluorophore that requires (FIG. 1A) close proximity of the fluorophores, and (FIG. 1B) overlap the donor emission spectrum with the acceptor excitation spectrum. FIG. 1C depicts a schematic of showing that TR-FRET utilizes the long excitation lifetime of lanthanides where the lifetime TR-FRET Donor is identified as (+) and the Lifetime Background Fluorescence (*). The time delay between excitation and detection allows for virtual elimination of background signal. FIG. 1D depicts the excitation (*) and emission spectra (+) of CoraFluors exhibit a large effective Stokes shifts and discrete Tb-emission bands, rendering them compatible with many acceptors.

FIG. 2 Comparison of Lumi4Tb and CoraFluor. Modification of the linker-attachment site allows for shorter (22 vs 11 steps) synthesis, yielding TR-FRET donors with improved stability and physicochemical properties (e.g., CoraFluor). CoraFluors are also devoid of a chiral center, which in combination with the axial chirality of the octadentate ligand results in diastereomers for the Lumi4-type donors.

FIGS. 3A-3C TR-FRET distance extension. FIG. 3A is a schematic of showingsize comparison of representative biochemical structures. The red circles designated with (*) illustrate the size of space that can be probed efficiently with TR-FRET-based techniques. While binding events that are at greater separation, e.g., binding to distal sites of the ribosome, cannot be interrogated with current TR-FRET techniques. FIG. 3B is a schematic of a stationary model, that show that even at high concentrations, donor and acceptor molecules are not within productive FRET distance unless specifically bound. FIG. 3C is a schematic that shows that the longer the excitation lifetime of the donor, the more likely it is for an excited donor to diffuse within R₀ of an acceptor, resulting in FRET signal from unbound acceptors.

FIG. 4A shows the slow excitation of lanthanides. When exciting with continuous light sources, maximum excitation is only achieved after longer light pulses (shaded box).

FIG. 4B shows normalized emission of terbium, dysprosium, europium, and samarium.

FIG. 5 shows compounds that contain modifications to the CoraFluor ligand, including changes to top and bottom by formally replacing the central ethylene diamine with urea, oxamide, and conformationally restricted diamines. The modification to the isophthalate ligand probes halogenation and alkyl substituents. The experimental strategy to access the required intermediates is outlined in FIGS. 6A-6C.

FIGS. 6A-6C shows the experimental strategy to access the intermediates.

FIGS. 7A-7C shows excitation-emission lifetime measurements. FIG. 7A Shows the customized in-house benchmark setup and schematic. FIG. 7B shows the platform that enables the sensitive measurement of excitation and emission kinetics with sub-microsecond resolution. FIG. 7C shows the luminescent decay of CoraFluor-lanthanide complexes (Tb, Eu, Dy, Sm) measured with the custom platform.

FIGS. 8A-8D depict TR-FRET microarrays. FIG. 8A shows aschematic illustration of microarrays. FIG. 8B shows that in a heterogenous system the surface can carry the TR-FRET acceptor (Design 1) or the TR-FRET donor (Design 2). This can be done by direct labeling of the immobilized component or installation of the acceptor/donor adjacent to the immobilized component. The soluble component is labeled with the respective TR-FRET partner. Binding occupancy is dependent on the bulk concentration. However, only ligands within short distance can diffuse from the bulk phase to the surface. FIG. 8C Shows that design 1 minimized signal derived from unbound ligand. In contrast an unbound tracer ligand that is close to the surface will pass by many donors during the excitation lifetime, resulting in high non-specific signal. FIG. 8D schematically shows heterogenous assay design concepts.

FIGS. 9A-9D depict heterogenous TR-FRET studies. Streptavidin immobilized to 384-well plates was labeled with Tfp-esters of CoraFluor-1 (FIG. 9A) and Alexfluor488 (FIG. 9B). Then 10 nM CoraFluor-1-desthiobiotin or TAMRA-desthiobiotin were added in the presence or absence of 100 μM biotin. TR-FRET signal was detected with a SPARK microtiter plate reader. FIG. 9C shows the nonspecific signal increases with increasing concentration of free acceptor ligand. FIG. 9D shows CoraFluor-1 was deposited in 3 μL in the well-center of a 96-well plate, and TR-FRET signal was measured with a SPARK microtiter plate reader set to scan a 5×5 matrix read covering the whole surface of the well.

FIG. 10A shows that a stock solution of 1 mM FITC and TAMRA were dispensed with an HP D300 on top of 384-well plate that had been covered with an adhesive seal (to better illustrate the pattern) with a 6144-well matrix with a maximum volume of 100 nL.

FIG. 10B shows a close-up view of FIG. 10A.

FIG. 10C shows that this design enables the accurate placement of a 5×5 matrix in a 96-well plate.

FIG. 11 shows a schematic illustration of array printed on well bottom and peptide microarray.

FIG. 12 contains a schematic representation of quantitative modeling for microarray applications. FIG. 12 depicts a schematic representation of quantitative modeling for microarray applications. Streptavidin is shown as a monolayer that would be distributed in a 30 μL solution corresponding to a 20 nL solution on a 384-well array format. The image of the 384-well array illustrates multiple binding sites for potential target molecule attachment. The 384-well plate includes a surface area/well of about 0.084 cm² with the number of protein molecules per well of about 3.36×10⁻¹³ mol with a nominal concentration of about 30 μL and about 20 nM (about 1.87×10−8 mol). The left portion of FIG. 12 depicts the 3D structure of a streptavidin protein with its respective Protein Data Bank (PDB) identifier 1SWA. Such streptavidin proteins are homotetramer of 60 kDa with dimensions of about 4.2 nm×4.2 nm×5.6 nm with a cross-section of about 25 nm² and the monolayer of the plate is about 4 μmol/cm². FIG. 12 illustrates the transformation of the volume of a solution to the surface area of a microarray and how a protein's structure can be represented and studied in this environment. The bead formed is about 5-6 μM with a surface area of about 1×10⁻⁶ with the number of protein molecules/bead is about 2.5×10⁶.

FIG. 13 contains schematic representation of FRET microarray reader compared to traditional microarray scanner. The left side of FIG. 13 depicts a traditional microarray scanner. The traditional microarray scanners generally need raster imaging and are slower with increased susceptibility to background. The right side of FIG. 13 depicts the TR-FRET microarray disclosed herein. The TR-FRET microarray reader includes functionality having less expensive optics, and simultaneous and continuous imaging of a larger area compared to traditional microarray solutions. The TR-FRET array includes confocal optics which can reject out-of-focus signals (e.g., microfluidics). The TR-FRET microarray reader disclosed herein can enable kinetic profiling (e.g., microfluidics). Compared to a traditional microarray scanner, the TR-FRET microarray reader allows for the equivalent of massively parallel surface plasmon resonance (SPR)/Bio-layer interferometry (BLI).

FIG. 14 contains a schematic representation of FRET microarray readers built on existing platforms. Examples of existing platforms depicted in FIG. 14 include a FluorChem and a Luminex MagPix device.

FIG. 15A contains a schematic representation of Representative Example—Kinase Microarray 1. The left image of FIG. 15A includes individual kinase isoforms (e.g., >500) arrayed on a slide. The acceptor is globally installed on proteins or slide surfaces. The middle image depicts CoraFluor-labeled pan-kinase ligand (e.g., staurosporine) that can bind to all kinases. The right image of FIG. 15A depicts the addition of a specific kinase inhibitor of interest. The kinase inhibitor will displace donor ligand depending on affinity for a respective kinase.

FIG. 15B contains a schematic representation of Representative Example—Kinase Microarray 2. The left image of FIG. 15B includes individual kinase isoforms (e.g., >500) arrayed on a slide, acceptor globally installed on proteins or on slide surface. In the example shown by FIG. 15B, CoraFluor-labeled kinase ligand of interest. The right image of FIG. 5B depicts the example kinase microarray ligand that will only bind to kinases in a dose-dependent fashion.

FIG. 15C contains a schematic representation of Representative Example—Histone-tail Peptides 1. The left image of FIG. 5C depicts individual histone peptides with and without post-translational modifications arrayed on a slide with acceptors globally installed on proteins or a slide surface. CoraFluor-labeled epigenetic reader (e.g., bromodomain protein) is depicted in the middle. The right image of FIG. 15C depicts the ligand binding to the specific histone tail modification in a dose-dependent fashion. In some embodiments, the example histone-tail peptide of FIG. 15C is can be translated to inhibitor screening.

FIG. 15D contains a schematic representation of Representative Example—Histone-tail Peptides 2. The left image of FIG. 5D depicts individual histone peptides with and without post-translational modifications arrayed on a slide with acceptors globally installed on proteins or on a slide surface. FIG. 5D depicts the example with an epigenetic writer (e.g., acetyl or methyl transferases) (top) and an epigenetic eraser (e.g., deacetylases or demethylases) (bottom). The right image of FIG. 5D depicts the example histone-tail peptide as recognition by context-independent or dependent antibody (e.g., anti-acetyl lysine).

FIG. 15E contains a schematic representation of Representative Example—Ligase Substrate Characterization. The left image of FIG. 15E depicts a human proteome array with the acceptor globally installed on proteins or slide surfaces. FIG. 5E depicts a ligase enzyme (e.g., kinase) and the right image depicts the example ligase substrate characterization recognition by antibody (e.g., anti-phospho-tyrosine antibody).

FIG. 15F contains a schematic representation of a representative example—DNA-Encoded Library (DEL) SAR Array. DNA-encoded small molecule libraries haven been widely used for the discovery of bioactive small molecule ligands. The ability to establish comprehensive quantitative binding information on a larger compound set is inherently limited. (TR-) FRET-based microarray approaches utilizing established oligonucleotide platforms will allow to directly array DELs with densities of 10⁴-10⁶ compounds per array and directly establish complete SAR for the small-molecule library.

FIG. 16A shows the results of the FRET microarray with FRET from surface to bound molecules. Streptavidin-coated 384-well microtiter plate labeled covalently with 25 μM AF488-Tfp ester for 30 minutes. The unreacted reagent was washed away with 5× washes. Blocked half wells with 100 μM biotin for 20 minutes. Added 10 nM CoraFluor-1-HaloTag-Desthiobiotin trifunctional probe for 1 hour. Then, read the TR-FRET signal. The results are shown in FIG. 16A that a surface can be with a TR-FRET acceptor and measure specific FRET signal from the bound TR-FRET donor molecule to the acceptor-labeled surface. In this case, the signal from the bound TR-FRET can be competed with biotin. Overall, the results from FIG. 16A shows proof of concept (POC) for a TR-FRET microarray where excess fluorophore does not need to be washed off before imaging and/or measurement.

FIG. 16B shows results of FRET microarray with FRET from surface to bound molecules. Streptavidin-coated 384-well microtiter plate labeled covalently with 25 μM CoraFluor-1-Pfp ester for 30 minutes. The unreacted reagent was washed away with 5× washes. Blocked half wells with 100 μM biotin for 20 minutes. Added 10 nM TAMERA-HaloTag-Desthiobiotin trifunctional probe for 1 hour. Then, read the TR-FRET signal. These results show that a surface can be labeled with a TR-FRET donor and specific FRET signals can be measured from the surface-bound acceptor molecule. Overall, the results from FIG. 16A shows POC for a TR-FRET microarray where excess fluorophore does not need to be washed off before imaging and/or measurement.

FIG. 17A contains a schematic showing applications of FRET is Novel Time-gated microfluidics platforms. FIG. 17A shows an example TR-FRET microarray reader system that significantly enhances photodetector performance by mitigating background signals, which is a common limitation in many instruments. Utilizing a flow cell, background interference is effectively addressed by a time-delayed measurement technique. Moreover, the system capitalizes on the intrinsic advantages of TR-FRET signal detection, which relies on a time-resolved approach markedly distinct from conventional fluorescence or time-resolved fluorescence methods. FIG. 17B contains a schematic showing integration of time-gated microfluidics with multiplexing. FIG. 17B depicts the enhanced multiplexing capabilities of a TR-FRET detection system as disclosed herein. Embedding TR-FRET functions into many targets allows for substantial scalability in multiplexing. Specifically, the system can achieve exponential increases in detection channels when combining distinct channels; for example, combining 100×100, 500×500, or 1000×1000 channels yields 10,000, 250,000, or 1,000,000 distinct channels, respectively. This approach expands the encoding potential, with the 1000×1000 combination providing up to one million unique channels. The estimation suggests that the system can facilitate the parallel detection of numerous targets, establishing a new benchmark in multiplexed assay capabilities.

FIG. 17C contains a schematic description of how Luminex xMAP INTELLIFLEX can be useful as a time-gated microfluidics platform. FIG. 17C outlines the use of a Luminex xMAP INTELLIFlex analyzer for dual-wavelength experiments. Although the 405 nm laser is not ideal for this purpose, reports suggest that the intensity is comparable to that of a 532 nm laser when used with DAPI stains, which can be used for nuclear staining. The demonstrates the concept's viability and the potential improvements in signal intensity and detection sensitivity through the optimization of next-generation laser excitation.

### DETAILED DESCRIPTION

Proximity is arguably the ultimate master regulator of life. On a molecular level, the interactions of biomolecules with each other and/or with small-molecule ligands control virtually all aspects of human health and disease. Studying the interaction between biomolecules, or biomolecules with small molecule ligands, is at the very core of chemical biology. Most translational biomedical research programs—in academia and industry—focus on developing therapeutic strategies that modulate these events. Here, progress and success are highly dependent on the availability of robust, quantitative, and sensitive assay platforms that allow for straightforward but accurate characterization of the underlying molecular interactions or the measurement of target abundance. Thus, techniques that enable scientists to interrogate and quantify these events reliably, preferably in their natural environment, are indispensable for life-science research. Ideally, such assays can be performed using standardized high-throughput screening (HTS) compatible formats that are easy to handle and that do not require any highly specialized capital equipment, which is generally not available in a typical research group or department infrastructure and too expensive to “just buy” (a consideration that is particularly critical for academic groups). Furthermore, such assays should be easily implemented and directly compatible with the cell lines and tissues most relevant to the research project. In this regard, fluorescence-based technologies play a central and dominant role in biomedical research and clinical diagnostics. Most high-throughput assays are based on various fluorescence detection modes due to their high sensitivity, large dynamic range, signal stability, variety of readily accessible fluorophores, and ease of operation. Similarly, fluorescence-based microscopy techniques comprise arguably the most important imaging technology currently employed in biomedical research. In recent years, the application of time-resolved (TR) fluorescence (TRF) measurements has dramatically improved the sensitivity of homogenous biochemical assays and high-resolution microscopy. Furthermore, coupling TR readouts with Förster resonance energy transfer (FRET) offers exceptional sensitivity and specificity. TR-FRET assays are widely appreciated for their superior performance in HTS, including the measurement of ligand binding or displacement, protein-protein/lipid/nucleic acid/carbohydrate interactions, enzyme activity, gene expression, protein secretion, and antibody profiling, amongst others.

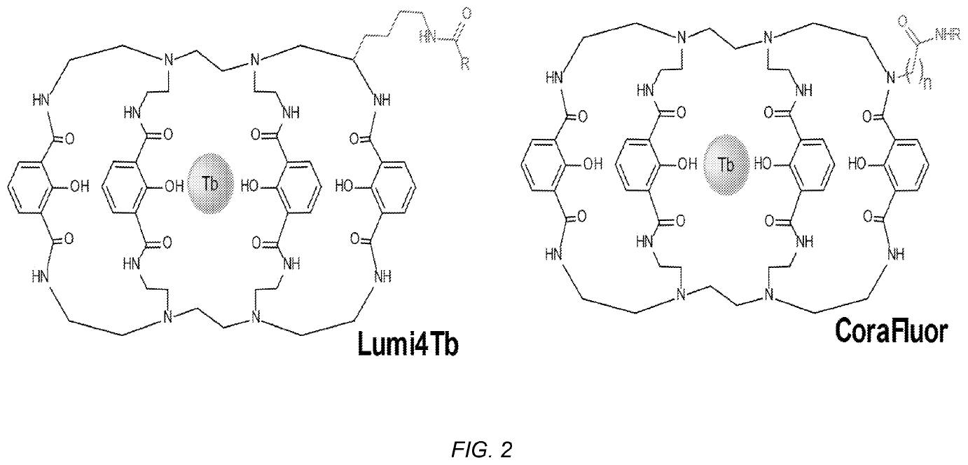

In TR-FRET-based assays, a signal is generated through FRET between a donor with a long luminescence lifetime and an acceptor fluorophore when in close proximity to each other (5-10 nm, approximately the size of a nucleosome). The time-gated measurement allows for the virtual elimination of nonspecific background signals originating from scattered excitation light and autofluorescence of screening compounds, reagents, and assay plates. At the same time, the FRET component limits the readout to acceptor molecules in the donor's immediate proximity. This approach, therefore, enables the quantitative measurement of the interaction of biomolecules and/or small molecule ligands with superior sensitivity. Important for the development of sensitive and robust TR-FRET assays is the availability of fluorescence donors that satisfy the following criteria: 1) stable in biological buffers, 2) sufficiently long luminescence lifetime, 3) good quantum yield and brightness, 4) insensitive to assay environment, and 5) scalability and costs. 6) In addition, for cell-based assays, the donors must efficiently diffuse through cellular membranes. The favorable luminescent properties of lanthanide (Ln)-based emitters (long lifetimes, narrow luminescent bands, large effective Stokes shifts) make them uniquely attractive as FRET-donors for a variety of biomedical applications. Of particular relevance are complexes containing Tb (terbium) or Eu (europium), and to lesser extend Sm (samarium) or Dy (dysprosium). Specifically, highly coordinated cryptate complexes of these metals have been recognized for their desirable properties. While hundreds of luminescent Ln-complexes have been reported, only a small number satisfy the characteristics required for biological assay and imaging applications. Just a few of these reagents are commercially available. The precise chemical structures of some of the commercial reagents are not disclosed, and published structures generally require a lengthy, inflexible, and challenging synthesis, which limits accessibility to these reagents. Commercially available products are priced at a premium. Lumi4Tb (CisBio, PerkinElmer) is the gold standard and the only FRET-donor compatible with most assay conditions (FIG. 2). However, the high costs (about $500,000 per milligram), which translates to reagent expenses of $1-10/well, or the demanding 22-step synthesis significantly limits access to these reagents, in particular for academic labs and makes it prohibitive for experiments that would require milligram quantities for further chemical functionalization of the ligand. This, in turn, has hampered the development and optimization of these reagents for new applications. However, it would be desirable to translate this powerful technology more broadly to cell-based and even in vivo applications. Unfortunately, the physicochemical and photophysical properties of existing ligands are not well suited for these applications. Lumi4Tb and other TR-FRET donors are not membrane-permeable, and the required excitation wavelength of about 340 nm is generally unavailable on laser-based microscopy equipment. Therefore, TR-FRET applications have been relegated primarily to homogenous assay platforms and assays for cell surface receptors, leaving bioluminescence resonance energy transfer (BRET) as the only viable option. While potential advantages of BRET include genetic encoding of the energy transfer donor and obviating the need for an excitation light source, BRET ultimately depends on the activity of a luciferase for signal generation and is therefore confined by several fundamental limitations inherent to enzymatic reactions. Besides the need for continuous supply of a substrate, the luciferase activity requires oxygen and is affected by temperature, pH and ionic strength, amongst others. Consequently, BRET is incompatible with low and high temperatures, unsuitable for anaerobic processes, and sensitive to pH and salt concentration. The turnover rate of the luciferase limits total signal intensity, and the emission spectrum limits the number of compatible acceptor fluorophores, which restricts the ability to multiplex. In contrast to TR-FRET, which allows for tight, spatiotemporally controlled excitation of the donor fluorophore, BRET also does not provide meaningful control in these dimensions. Furthermore, the Förster radius of lanthanide TR-FRET pairs is generally larger than that afforded by both conventional FRET and BRET, which enables the probing of larger protein complexes. Lastly, the requirement for the productive spatial orientation of donor and acceptor transition dipoles can limit the use of BRET and require lengthy optimization of the linker employed for luciferase fusion protein constructs. As Ln-donor emission is not polarized as in BRET, TR-FRET provides greater topological flexibility between donor and acceptor.

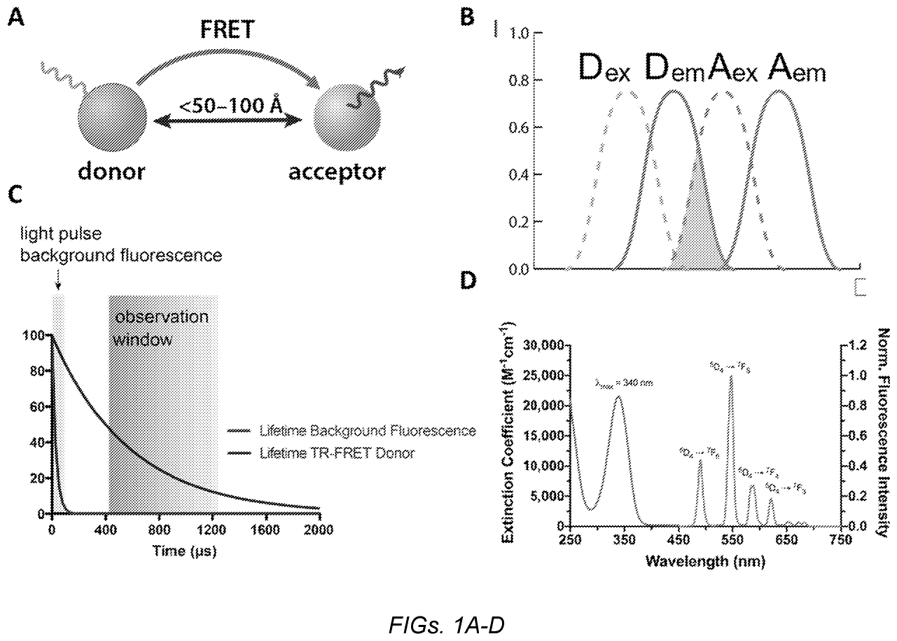

One example of FRET donor useful for the microarray approaches provided in this disclosure is CoraFluors, which rely on a non-obvious yet conceptually simple and impactful structural modification (FIG. 2). The change of the linker attachment site (compared to Lumi4Tb acceptor) to an amide nitrogen, which was assumed to be incompatible with the function and synthetic methodology, are not only renders CoraFluors unencumbered by prior art and therefore unrestricted for academic research, but they also resolve many of the existing shortcomings of current TR-FRET donors, and they are accessible through a robust and modular synthetic strategy that provides facile access on >100 mg scale. CoraFluors tolerate a wide range of biological conditions and exhibit superior photophysical and physicochemical properties compared to commercial competitors. Furthermore, they are readily compatible with most tracers of existing FRET, BRET, and FP platforms, enabling seamless integration while providing improved assay performance (FIG. 1D). Importantly, CoraFluors are not only best in class but also the first cell permeable TR-FRET donors and are suitable for real-time quantification of specific target binding of small molecule inhibitors in live cells.

### FRET Distance for Microarray Platforms

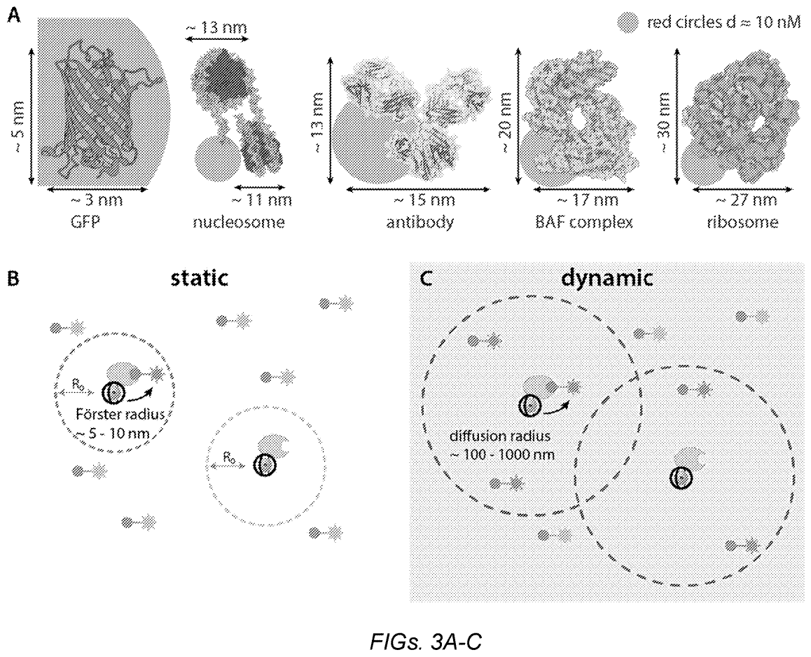

The efficiency of FRET processes (which include BRET) is >50% for acceptors located within the Förster radius (R₀) of the donor but declines proportionally to the inverse sixth power beyond R₀ (i.e., the intensity drops to about 1% at about 2×R₀). For small molecule FRET donors, R₀ is typically 5-10 nm. Although R₀ tends to be bigger for TR-FRET systems than regular FRET and BRET processes, it is insufficient for probing larger biological structures, such as multiprotein complexes, functional assemblies, chromatin-associated complexes, or the ribosome, which are on the order of 20-30 nm (FIG. 3A). CoraFluors allow to take advantage of “diffusion enhanced FRET” which is a mostly undesired phenomenon in TR-FRET that can become problematic at high donor/acceptor concentrations (>10 μM). In a quasi-static view, as it applies to the nanosecond timescales of regular FRET and BRET processes, where molecules are locked in space, the mean distance between donor and acceptor is >R₀, even at 1 mM (FIG. 3B). However, with increasing concentration and longer luminescence lifetime of the donor, the probability increases for donors and acceptors to diffuse within R₀ during the excitation lifetime of the donor (the mean diffusion distance of a small molecule within 2 ms, the excitation lifetime of CoraFluor-1, is ~ 1,400 nm), allowing the FRET process to take place (think getting zapped by static electricity when about to touch a door knob) and thereby resulting in nonspecific background signal (FIG. 3C).

### Optophysical and Physicochemical Properties of FRET Pairs

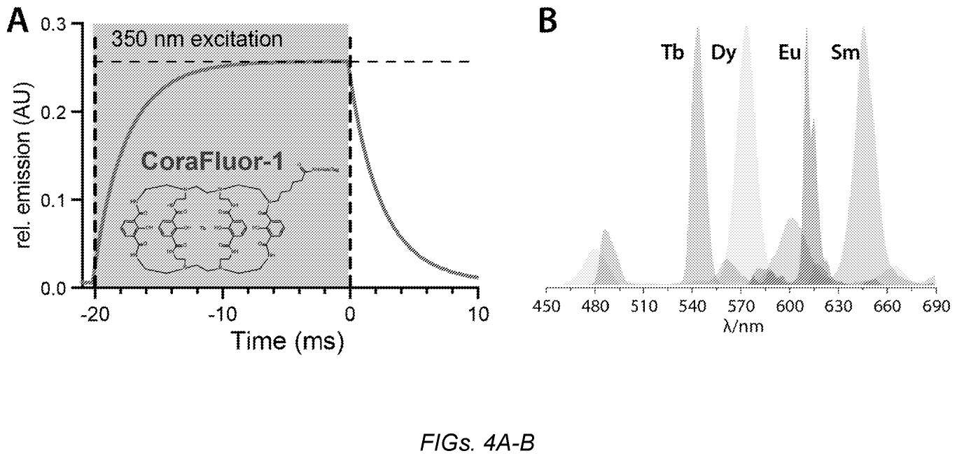

The CoraFluor (and Lumi4) ligands bind Tb and other lanthanides tightly in a quasi-centrosymmetric octadentate complex. However, while the ligands sensitize Tb with high quantum yields, they are highly inefficient for sensitizing Eu, Sm, or Dy. This is because the coordination symmetry and the characteristics of the N—H oscillators directly affect the photophysical properties of these complexes, including the radiative rate constant. In highly symmetric complexes, the desired excitation processes are slow because they represent Laporte-forbidden 4f-4f transitions, and for non-Tb lanthanides, other non-radiative processes dominate, resulting in low quantum yields. Similar considerations are also responsible for the slow excitation kinetics, which we can accurately characterize with custom-built profiling platform. This rarely recognized characteristic becomes apparent when a complex is not excited using high-powered pulsed lasers or microsecond xenon flash lamps but using lower-powered continuous light sources (e.g., LED s) that are commonly used in microscopes or low-cost analytical equipment (see FIG. 4A). Only a small population of the complexes can be excited at a given time, resulting in low signal output. Improving the excitation efficiency greatly enhances the sensitivity in virtually all applications. However, the same quantum mechanical principles do not apply to asymmetric complexes. By breaking the symmetry, e.g., by introducing asymmetric elements or deliberately decreasing the coordination geometry of the lanthanide ion, the transition probabilities and, by extension, the radiative rate constant are greatly enhanced. This strategy is also suitable for the sensitization of Eu, Sm, Dy, which would allow for improved multiplexing and access to the near IR and IR spectrum, which is important for the translation to in vivo applications (FIG. 4B). CoraFluor-2 exhibits characteristics that yield an efficiency comparable to DAPI and Hoechst nucleic acid stains when excited with a 405 nm (~2-3% of λₘₐₓ) light source. For Tb-based complexes, inherent photophysical properties predict a theoretical upper limit of about 445 nm. Several Alexa Fluors and coumarin-based dyes, such as Alexa Fluor 405 and Coumarins 1 and 30, are suitable for this range and will be explored as conjugates. The linker-modified CoraFluors, such as those developed by the instant inventors previously, enable direct conjugation of the corresponding dyes to evaluate the performance.

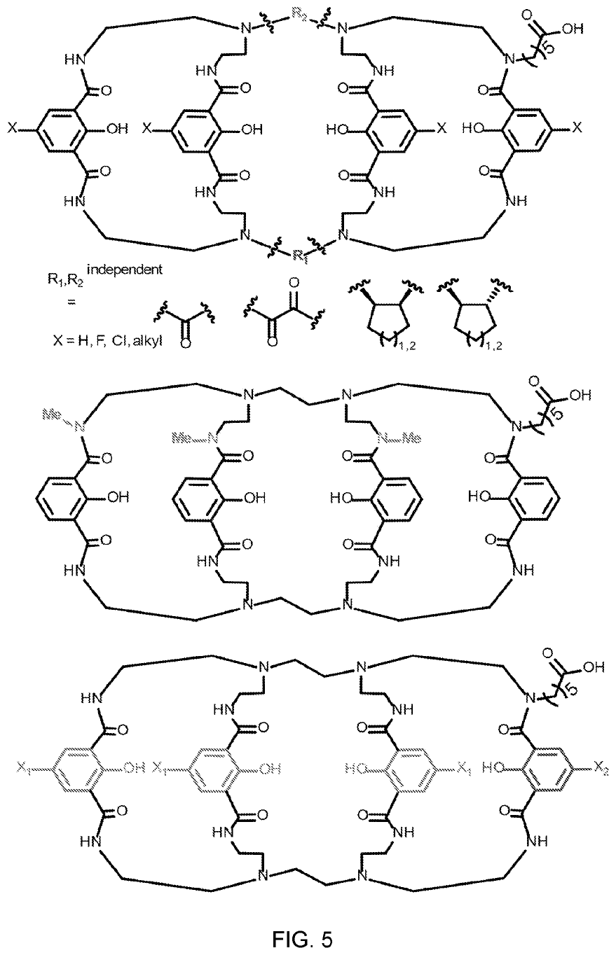

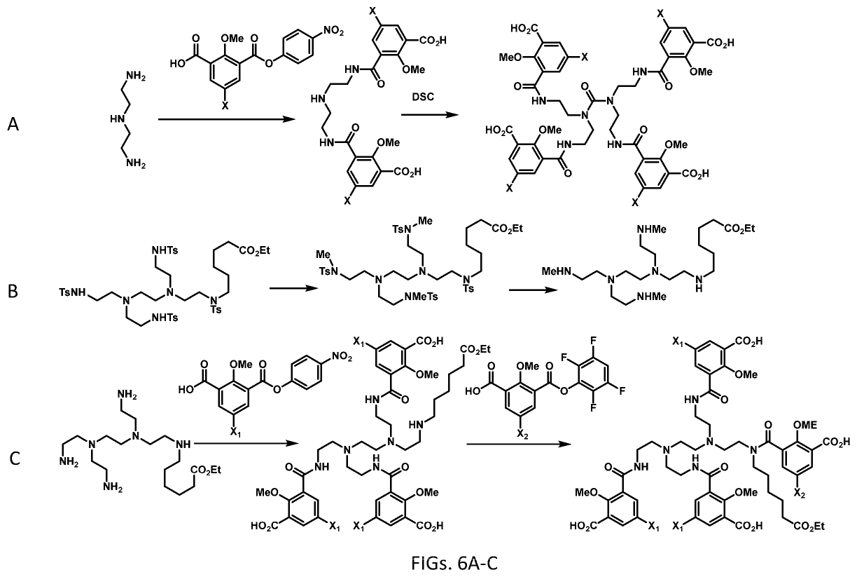

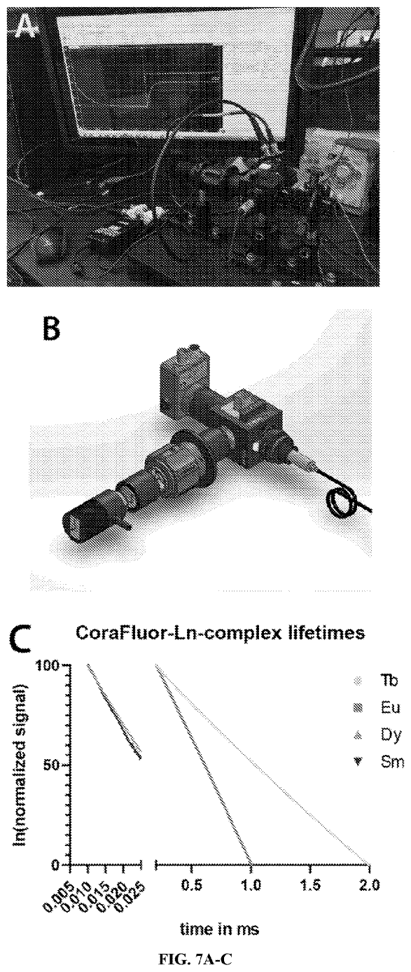

As described herein, the present disclosure provides various FRET acceptors including CoraFluor analogs with varying photophysical and physicochemical properties for the corresponding lanthanide complexes. Selected examples are illustrated in FIGS. 5 and 6A-C and include modifications to the tetra amine “cap” of the ligand. Examples include derivatives featuring a urea or oxamide in the central position. This design “eliminates” two (or four, if used for top and bottom) basic nitrogens and thereby change the overall polar character and logD, which increases membrane permeability (The logD of HT-CoraFluor-2 is 0.7, which is close to the optimum range of 1-3). Furthermore, if only applied to one side, this modification break the quasi-centrosymmetric structure and sensitize Eu, Sm and Dy more efficiently. Exemplary compounds include electron-donating and electron-withdrawing substituents on the isophthalic acid moieties, which directly impact the lifetime and absorbance spectra. Aliphatic modifications do not change the electronic properties but allow the appendage of functional groups that alter the charge, solubility, logD, cellular distribution, and polarity. Lipophilicity of the ligand can be altered by isophthalate building blocks with short alkyl substituents (C2-C5), which are readily accessible from the corresponding bromo-substituted starting material via Sonogashira-coupling followed by hydrogenation. These features can be used to append “antenna” molecules. This provides access to non-centrosymmetric ligands. Ligands described herein are loaded with Tb, Eu, Dy, Sm, and characterized for their luminescent properties.

### TR-FRET-Based Assay Methodologies

Driven by the ever-increasing demand for higher throughput, increased sensitivity, simpler workflows, and improved robustness, assay technology has advanced significantly over the past decades. However, satisfying these demands has often come with other challenges-higher reagent costs and the requirement for specialized expensive equipment. Though, the extent to which scientific progress can be accelerated by new technologies that decrease costs while improving throughput and simplifying the workflow is nowhere better illustrated than by advances in gene sequencing technology. An ideal assay platform enables the direct and quantitative measurement of the interaction between a ligand (e.g., small molecules, peptides, proteins, or other biomolecules such as carbohydrates, lipids, and nucleic acids) with a target protein of interest (POI). Preferentially such a platform offers the flexibility to determine both thermodynamic and kinetic binding constants.

For screening approaches that aim to identify novel ligands for a POI or seek to comprehensively understand the selectivity of a given POI for a ligand class, it is desirable to perform such experiments in high throughput. For target engagement studies, most TR-FRET strategies employ donor-labeled antibodies that are directed against epitope tags (e.g., His6-tag, GST-tag, FLAG-tag, HaloTag), labeled (strept) avidin for biotinylated proteins/peptides, or TR-FRET ligands for functionalization of self-labeling protein tags (e.g., HaloTag, SNAP-tag). However, sometimes the expression of epitope-tagged fusion proteins is unsuccessful, or the epitope tag can interfere with the protein function. In these instances, it can be necessary to assay the native, unmodified wild-type POI. Described herein are TR-FRET assay approaches that extend the applicability beyond current assay formats. The systems within the instant claims translate the advantages of TR-FRET technology to microarray platforms.

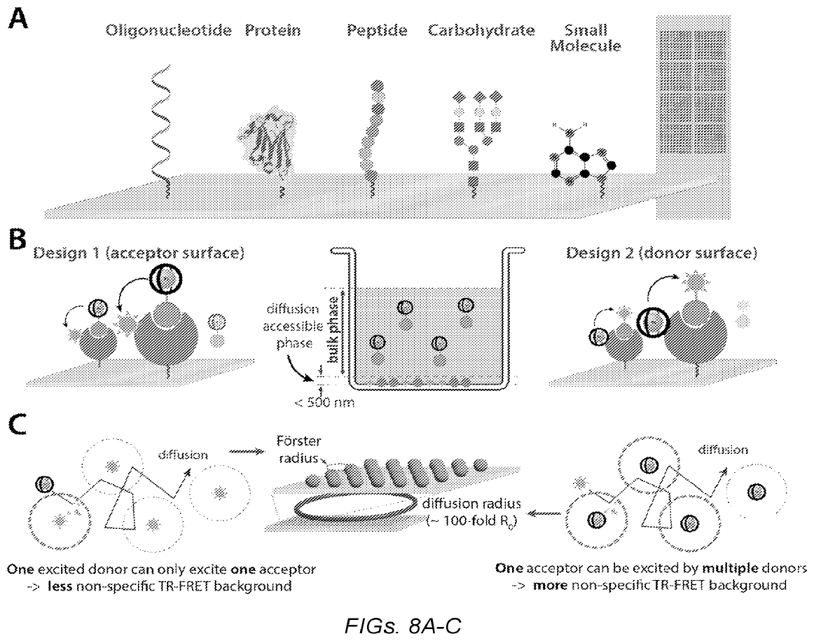

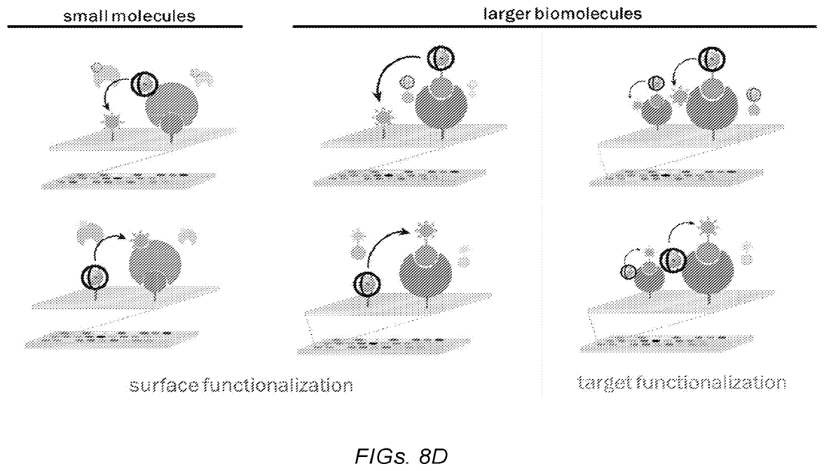

Conceptually, array-based technologies represent a promising high-throughput platform for profiling large numbers of immobilized molecules for target or substrate recognition. As briefly discussed above, all of these approaches follow the same canonical scheme: libraries of the respective target molecules (e.g., DNA, RNA, proteins, peptides, small molecules, carbohydrates) are immobilized or directly synthesized on a solid substrate (e.g., surface-treated or surface-coated glass or silicon slides) in a defined spatially-encoded 2-dimensional array (FIG. 8A). Then the microarray is incubated with a solution containing the fluorescently labeled analyte(s) of interest, allowing the analyte to bind. The major drawback is the requirement for extensive wash-steps to remove the unbound ligand to minimize nonspecific signal before readout. This approach works well for specific and high-affinity interactions, such as oligonucleotide arrays, that are compatible with extensive washing protocols. However, lower affinity interactions, e.g., as typical for small-molecule and other non-oligonucleotide microarrays, are often incompatible with extensive washing protocols and, therefore, much more challenging to capture with this technique.

TR-FRET approach described herein overcomes the inherent limitations of traditional microarrays and, in addition, enables quantitative thermodynamic and kinetic measurements. Since TR-FRET rejects nonspecific background signal, it is compatible with wash-free experimental designs (as it is done routinely in homogenous assays). This, in turn, enables to a) capture low-affinity interactions, and measure b) concentration-dependent, and c) time-dependent binding studies, which allows the massively parallel determination of equilibrium binding constants and binding kinetics.

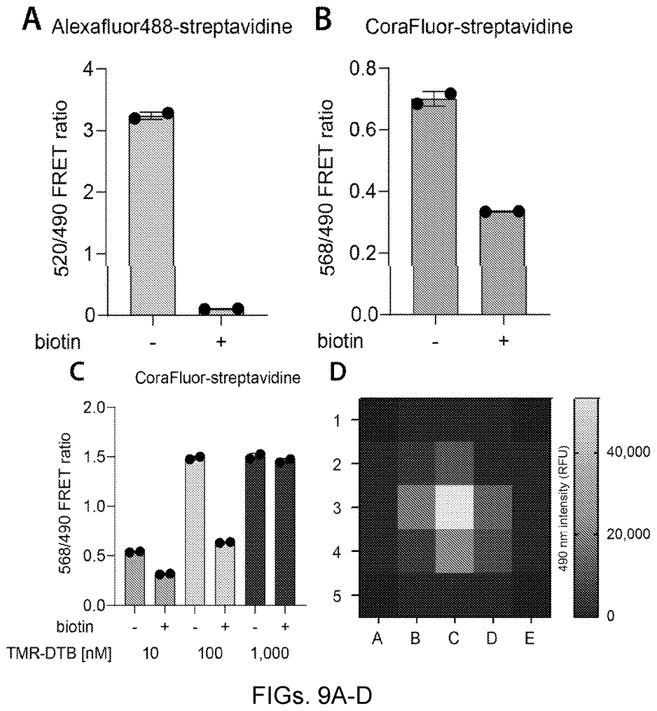

Although heterogeneous assays can be less desirable than homogenous assays for various reasons, one of the most frequent issues is related to nonspecific background. TR-FRET-based heterogeneous approaches, which are equally applicable to array and plate-based implementations, offer a previously unrecognized feature that can be exploited to improve assay performance substantially (FIG. 8B). In a heterogeneous experimental setup, the surface-immobilized component can be directly or adjacently tagged with the TR-FRET acceptor, with the unbound analyte labeled with the TR-FRET donor (Design 1). Alternatively, the donor and acceptor can be switched (Design 2). However, these designs are not equivalent. Most of the unbound soluble components will be in the bulk phase and unable to diffuse to the surface within the excitation lifetime. Since one acceptor can harvest energy from multiple donors, but a donor can only excite one acceptor, Design 1 limits the nonspecific signal because an unbound ligand can only excite one acceptor (FIG. 8C). In contrast, due to the very short excitation lifetime of the acceptor (2-20 ns) it can be excited by many donors when diffusing in proximity to the surface (Design 2), resulting in significant background contribution (FIG. 8C). Experimental data supports the surprising and unexpected advantage of design 1 over design 2. Streptavidin-coated 384-well plates were lysine-labeled with AlexaFluor488 or CoraFluor-1-Pfp ester. Then added in the presence or absence of excess biotin 10 nM CoraFluor-1-desthiobiotin or TAMRA-desthiobiotin. As shown in FIGS. 9A-D, the acceptor-surface modified strategy, offered far superior signal-to-background. Further consistent with this model, increasing the concentration of free TAMRA-DTB results in a significantly increased background signal that is dominant at 1 μM (FIG. 9C, 10A-C, 11).

### Exemplary Embodiments

In some embodiments, the present disclosure provides assays for studying interactions between a target molecule and a ligand molecule. For example, the assay can be used to determine presence or absence of binding between the target molecule and the ligand molecule (e.g., the presence or absence of a target molecule on a surface of a slide). In another example, the assay can be used to characteristics of the interactions between the target molecule and the ligand molecule. In an example wherein the ligand molecule is a protein of interest, the assay can be used to identify novel modulators of the protein, for example, using a target displacement strategy (e.g., where a novel modulator may be displacing interaction of the original target molecule and the ligand molecule).

Accordingly, in some embodiments, the present disclosure provides for detecting an interaction of a target molecule and a ligand molecule, the method comprising:
(i) treating a surface modified with a target molecule, wherein either the surface or the target molecule is further modified with a moiety which is a first member of the FRET acceptor/FRET donor pair, with a ligand molecule, wherein the ligand molecule is modified with a second member of the FRET acceptor/FRET donor pair that is complimentary to the first member (e.g., if the first member is a FRET acceptor then the second member is a FRET donor, and if the first member is a FRET donor then the second member is a FRET acceptor); and
(ii) exciting the surface with a light of a wavelength capable of being absorbed by the FRET donor moiety, waiting an amount of time sufficient for energy transfer from the FRET donor moiety to the FRET acceptor moiety, and detecting a light of a wavelength emitted by the FRET acceptor moiety by a fluorescence imaging technique.

In some embodiments, the surface is modified with a first member of the FRET acceptor/FRET donor pair. In some embodiments, the target molecule is modified with a first member of the FRET acceptor/FRET donor pair (e.g., the surface is not modified or not substantially modified with the first member).

In some embodiments, the present disclosure provides a method for detecting an interaction of a target molecule and a ligand molecule, the method comprising:
(i) treating a surface y modified with a target molecule, wherein either the surface or the target molecule is further modified with a FRET acceptor moiety, with a ligand molecule, wherein the ligand molecule is modified with a FRET donor moiety; and
(ii) exciting the surface with a light of a wavelength capable of being absorbed by the FRET donor moiety, waiting an amount of time sufficient for energy transfer from the FRET donor moiety to the FRET acceptor moiety, and detecting a light of a wavelength emitted by the FRET acceptor moiety by a fluorescence imaging technique.

In some embodiments, the surface is modified with the FRET acceptor moiety. In some embodiments, the target molecule is modified with FRET acceptor moiety (e.g., the surface is not modified or not substantially modified with the FRET acceptor moiety).

In some embodiments, the present disclosure provides a method for detecting an interaction of a target molecule and a ligand molecule, the method comprising:
(i) treating a surface modified with a target molecule, wherein either the surface or the target molecule is further modified with a FRET donor moiety, with a ligand molecule, wherein the ligand molecule is modified with a FRET acceptor moiety; and
(ii) exciting the surface with a light of a wavelength capable of being absorbed by the FRET donor moiety, waiting an amount of time sufficient for energy transfer from the FRET donor moiety to the FRET acceptor moiety, and detecting a light of a wavelength emitted by the FRET acceptor moiety by a fluorescence imaging technique.

In some embodiments, the surface is modified with the FRET donor moiety. In some embodiments, the target molecule is modified with the FRET donor moiety (e.g., the surface is not modified or not substantially modified with the FRET donor moiety).

In some embodiments, the present disclosure provides a method of identifying a compound (e.g., a test compound) that modulates a protein of interest, the method comprising:
(i) treating a surface modified with a protein of interest, wherein either the surface or the protein of interest is further modified with a FRET acceptor moiety,
with a ligand molecule, wherein the ligand molecule is modified with a FRET donor moiety (e.g., where the ligand molecule is a known modulator of the protein of interest);
(ii) exciting the surface with a light of a wavelength capable of being absorbed by the FRET donor moiety, waiting an amount of time sufficient for energy transfer from the FRET donor moiety to the FRET acceptor moiety, and detecting a light of a wavelength emitted by the FRET acceptor moiety by a fluorescence imaging technique;
(iii) treating the surface with a test compound;
(iv) after (iii), detecting a light of a wavelength emitted by the FRET acceptor moiety by a fluorescence imaging technique; and
(v) determining whether the intensity of fluorescence detected from the FRET acceptor moiety in step (iv) is decreased compared to the intensity of fluorescence detected from the FRET acceptor moiety in step (ii),
wherein said decrease in fluorescence intensity in an indication that the test compound is the modulator the protein of interest (e.g., the decrease in fluorescence intensity is indicative that the test compound displaces the interaction between the protein of interest and the ligand molecule).

In some embodiments, the compound is an inhibitor of a protein of interest (substrate-competitive orthosteric inhibitor, substrate-noncompetitive orthosteric inhibitor, or allosteric inhibitor). In some embodiments, the test compound is an antagonist or a partial antagonist of the function of the protein. In some embodiments, the compound is an activator of a protein of interest. In some embodiments, the test compound is an agonist or a partial agonist of the protein of interest.

In some embodiments, affinity of the ligand attached to the FRET acceptor/donor moiety to the protein of interest is less than affinity of the test compound to the protein of interest. For example, affinity of the ligand may be from about 100 to about 200 nM, while affinity of the test compound may be from about 10 nM to about 100 nM. In another example, affinity of the test compound is about 2×, about 4×, about 10×, about 20×, about 50×, about 100×, or about 200× greater compared to affinity of the ligand.

Step (iv) may be carried out in a manner similar to step (ii) above, by detecting and, if necessary, quantifying the fluorescence signal using a microscopy or spectroscopy device and associated software. The following step (v) of comparing the fluorescence intensity of step (iv) and the fluorescence intensity of step (ii) can be carried out using any suitable device or a piece of software. Without being bound by any particular theory, it is believed that the decrease in the intensity of fluorescence signal in step (iv) compared to step (ii) indicates that the test compound has bond to the protein of interest and thereby displaced the ligand, which is in turn indicative of the fact that the test compound is a modulator of the protein of interest. In some embodiments, the test compound has the same mode of action as the ligand (e.g., the test compound and the ligand are both inhibitors of the protein of interest). In other embodiments, the test compound and the tracer have different mode of action (e.g., the test compound is a substrate-competitive inhibitor and the ligand is allosteric inhibitor). In some embodiments, the fluorescence intensity in step (iv) is about 2×, about 4×, about 5×, about 10×, about 20×, about 50×, or about 100× less than in step (ii).

In some embodiments, any of the above method comprises (e.g., before the exciting of the surface in step (ii)) waiting a sufficient amount of time to allow for interaction between the target molecule and the ligand molecule (e.g., a protein of interest). In some embodiments, the sufficient time is from about 0.5 sec to about 10 min, from about 1 sec to about 1 min, from about 5 sec to about 1 min, or from about 10 sec to about 30 sec. In other words, the two components may be allowed to equilibrate for a period of time sufficient for the ligand to bind to the target molecule. For example, the surface can be equilibrated for about 10 min, about 30 min, about 1 hour, about 2 hours, or about 3 hours. The surface may be treated with a carrier liquid containing the ligand, such as water or a buffer solution to facilitate the binding. A concentration of the ligand in the solution may range from about 1 pM to about 1 μM, or from about 1 pM to about 1 nM.

In some embodiments, the surface is modified with the target molecule covalently For example, a linker is present between the surface and the target molecule that only contains covalent bonds. Examples of such linkers include polymers such as hydrophilic, hydrophobic, or amphiphilic polymers. Examples of covalent linkers linkers include poly(lactic acid-co-glycolic acid) and poly(ethylene glycol). The linker may also contain a moiety formed by a “click” reaction, such as a triazole. In some embodiments, the surface is modified with the target molecule non-covalently. In this example, the linker between the surface and the target molecule includes a non-covalent interaction such as H-bonding between base pairs of oligonucleotides, or streptavidin/biotin interaction, or an interaction between a hapten and an antibody. Any known method may be used to modify the surface with the target molecule or the FRET donor or FRET acceptor, including using known surface-modified chemistries and reactive groups, such as NHS, or maleimide functionalized microtiter plates and corresponding reagents to attach the target molecules. In some embodiments, the surface is a glass or a silicon slide. In some embodiments, the surface is a coated glass slide.

In some embodiments, in step (ii), said detecting of fluorescence may be carried out using fluorescent microscopy, fluorescent imaging probe, or fluorescent spectroscopy. In one example, both the excitation of the donor and detection (and measurement) of fluorescence of the acceptor can be performed using a single piece of equipment. Excitation can be carried out using a UV lamp or a laser. For detecting fluorescence, either photomultiplier (PMT) or charge-couple device (CCD) can be used to detect and quantify emitted photons. Also, total internal reflection fluorescence microscopy, light sheet fluorescence microscopy, or fluorescence-lifetime imaging microscopy can be used. The amount of time between exciting the FRET donor and reading fluorescence output from FRET acceptor can be from about 1 sec to 10 min, from about 5 sec and about 5 min, from about 10 sec to about 2 min, or from about 30 sec to about 1 min. In some embodiments, the wavelength of the light capable of being absorbed by the FRET donor moiety is from about 300 nm to about 400 nm. In some embodiments, the wavelength emitted by the FRET acceptor moiety is from about 450 nm to about 600 nm. A skilled analytical chemist would be able to tune the equipment as necessary depending to the particular excitation and fluorescent characteristics of the FRET pair used in the method.

In some embodiments, the target molecule is selected from small molecule, protein, peptide, nucleic acid, lipid, and carbohydrate. In some embodiments, the ligand molecule is selected from small molecule, protein, peptide, nucleic acid, lipid, and carbohydrate. In some embodiments, the target molecule is a small molecule and the ligand molecule is a protein. In some embodiments, the target molecule is a protein and the ligand molecule is a protein. In some embodiments, the target molecule is a peptide and the ligand molecule is a protein.

### FRET Acceptor Moieties

In some embodiments, the fluorescent FRET acceptor moiety is selected from fluorescein, AF488, hydroxycoumarin blue, methoxycoumarin blue, alexa fluor blue, aminocoumarin blue, Cy2 green (dark), FAM green (dark), alexa fluor 488 green (light), fluorescein FITC green (light), alexa fluor 430 green (light), Alexa fluor 532 green (light), HEX green (light), Cy3 yellow, TRITC yellow, Alexa fluor 546 yellow, Alexa fluor 555 3 yellow, R-phycoerythrin (PE) 480; yellow, Rhodamine Red-X orange, Tamara red, Cy3.5 581 red, Rox red, Alexa fluor 568 red, Red 613 red, Texas Red red, Alexa fluor 594 red, Alexa fluor 633 red, Allophycocyanin red, Alexa fluor 633 red, Cy5 red, Alexa fluor 660 red, Cy5.5 red, TruRed red, Alexa fluor 680 red, and Cy7 red. The waivelength of emitted light (e.g., maximum of emittance) for each of these FRET acceptor moities is well-known in the literature. Depending on the acceptor chosen, the chemist can adjust the selection of FRET donor and the various parameters of the step (ii) process, e.g., to ensure that the energy can be efficiently transferred from the donor moiety or the acceptor moiety.

In some embodiments, the FRET acceptor moiety has formula:

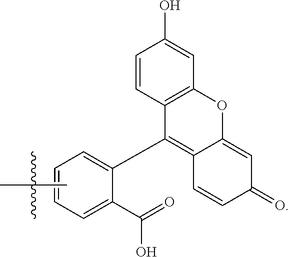

### FRET Donor Moieties

In some embodiments, the FRET donor moiety comprises a complex of a lanthanide metal with a moiety of formula (i):

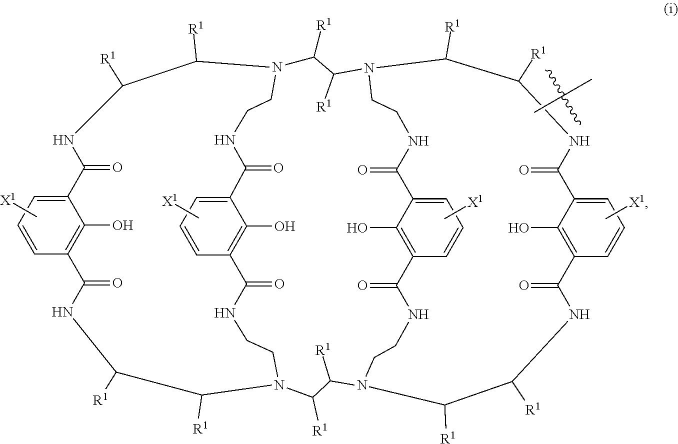

wherein:
each X¹ is independently selected from halo, NO₂, CN, N₃, C₁₋₆ alkyl, C₁₋₆ alkoxy, C₂₋₆ alkenyl, C₂₋₆ alkynyl, C₆₋₁₀ aryl, and 5-14 membered heteroaryl, wherein said C₁₋₆ alkyl, C₂₋₆ alkenyl, C₂₋₆ alkynyl, C₆₋₁₀ aryl, and 5-14 membered heteroaryl are each optionally substituted with 1, 2, or 3 substituents independently selected from halo, OH, SH, NH₂, C₁₋₃ alkylamino, di(C₁₋₃ alkyl)amino, NO₂, CN, C(O)OH, C₁₋₃ alkoxy, C₁₋₃ haloalkoxy, and N₃; and
each R¹ is independently selected from H, C₁₋₆ alkyl, C₂₋₆ alkenyl, and C₂₋₆ alkynyl, each of which is optionally substituted with 1, 2, or 3 substituents independently selected from halo, OH, SH, NH₂, C₁₋₃ alkylamino, di(C₁₋₃ alkyl)amino, NO₂, CN, C(O)OH, C₁₋₃ alkoxy, C₁₋₃ haloalkoxy, and N₃.

In some embodiments, ~ indicates a point of attachment of the donor moiety to either the protein of interest (e.g., to one of the side chains of an amino acid within the protein of interest), the antibody or nanobody, or the linker connecting the donor with protein, the antibody or nanobody, or a tag ligand, such as a halotag ligand. ~ also may indicate a point of attachment of the donor to streptavidin.

In some embodiments, each X¹ is independently a H or a halo.

In some embodiments, each X¹ is H.

In some embodiments, each X¹ is Cl or Br.

In some embodiments, one of R¹ is selected from C₁₋₆ alkyl, C₂₋₆ alkenyl, and C₂₋₆ alkynyl, each of which is optionally substituted with 1, 2, or 3 substituents independently selected from halo, OH, SH, NH₂, C₁₋₃ alkylamino, di(C₁₋₃ alkyl)amino, NO₂, CN, C(O)OH, C₁₋₃ alkoxy, C₁₋₃ haloalkoxy, and N₃; and the remaining R¹ groups are all H.

In some embodiments, each R¹ is H.

In some embodiments, the moiety of formula (i) has formula:

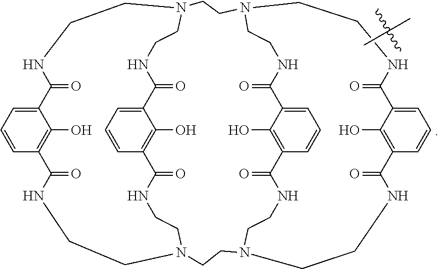

In some embodiments, the moiety of formula (i) has formula:

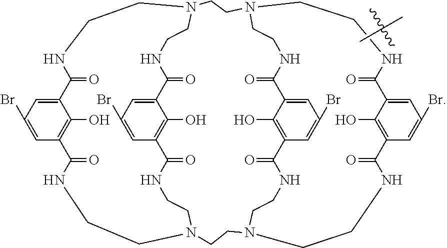

In some embodiments, the moiety of formula (i) has formula:

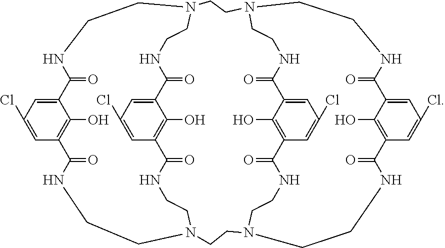 [SMILES: O=C1NCCN2CCNC(=O)c3cc(Cl)cc(c3O)C(=O)NCCN(CCNC(=O)c3cc(Cl)cc1c3O)CCN1CCNC(=O)c3cc(Cl)cc(c3O)C(=O)NCCN(CCNC(=O)c3cc(Cl)cc(c3O)C(=O)NCC1)CC2]

In some embodiments, the lanthanide metal is selected from Tb (terbium), Eu (europium), Sm (samarium), and Dy (dysprosium). In some embodiments, the lanthanide metal is Eu (europium). In some embodiments, the lanthanide metal is Sm (samarium). In some embodiments, the lanthanide metal is Dy (dysprosium). In some embodiments, the lanthanide metal is Tb (terbium). In some embodiments, the lanthanide metal is Tb³⁺.

In some embodiments, the target or the ligand is selected from an enzyme, a cell-surface receptor, nuclear hormone receptor, a transporter, a G-protein coupled receptor, a CD marker, a voltage-gated ion channel, a nuclear factor, a nuclear receptor, a protein-protein or protein-peptide interaction domain, scaffolding protein, structural protein, transcription factor, chaperone, and assembly/disassembly factor. In some embodiments, the enzyme is selected from kinases, proteases, deacetylases, ATPases, GTPases, phosphatases, peptidases, synthetases, phosphorilases, and nucleosidases.

In some embodiments, the target or the ligand is selected from KEAP1 protein, bromodomain protein, and an aminoacyl tRNA synthetase. In some embodiments, the protein of interest is selected from KEAP1 protein, bromodomain protein, an aminoacyl tRNA synthetase, and a histone deacetylase (e.g., HDAC1, 2, 3, 4, 5, 6, 7, 8, 9, or 10).

In some embodiments, the method includes making the target or the ligand attached to a FRET donor or a FRET acceptor moiety by contacting the target or the ligand comprising a halotag with a FRET donor/acceptor moiety comprising a halotag ligand. For example, the target or the ligand can be expressed as a fusion protein with halotag optionally with His6). In some embodiments, the method includes making the target or the ligand attached to a FRET donor/acceptor moiety by contacting the target or the ligand comprising an epitope tag with an antibody or nanobody to the epitope tag, the antibody or nanobody being attached to the FRET donor/acceptor moiety (directly or through a linker, e.g., a PEG linker).

In some embodiments, the method includes making the target or the ligand attached to a FRET donor/acceptor moiety by contacting the target or the ligand with an antibody or nanobody to the target or the ligand, the antibody or nanobody being attached to the FRET donor/acceptor moiety.

In some embodiments, the method includes making the target or the ligand attached to a FRET donor/acceptor moiety by contacting the target or the ligand with a first antibody or nanobody to the target or the ligand to obtain the antibody/nanobody conjugate, followed by contacting the conjugate with a second antibody or nanobody to the first antibody or nanobody, the second antibody or nanobody being attached to the FRET donor/acceptor moiety (directly or through a linker).

In some embodiments, the method comprises evaluating an interaction between the target molecule and the ligand molecule. In some embodiments, the fluorescence intensity detected from the FRET acceptor moiety is indicative of quality and/or quantity of the interaction between the target molecule and the ligand molecule.

In some embodiments, the method includes determining a thermodynamic binding constant between the target molecule and the ligand molecule.

In some embodiments, the method includes determining a kinetic binding constant between the target molecule and the ligand molecule.

In some embodiments, the method includes determining the mode of binding of the modulator to the protein of interest.

In some embodiments, the target molecule is a nucleic acid and the ligand molecule is a nucleic acid.

In some embodiments, the ligand molecule is a protein selected from an enzyme, a cell-surface receptor, nuclear hormone receptor, a transporter, a G-protein coupled receptor, a CD marker, a voltage-gated ion channel, a nuclear factor, a nuclear receptor, a protein-protein or protein-peptide interaction domain, scaffolding protein, structural protein, transcription factor, chaperone, and assembly/disassembly factor.

In some embodiments, the ligand/target/protein of interest in implicated in the disease or condition. Suitable examples of such compounds include proteins implicated in the pathology of cancer. Suitable examples of such proteins include kinases (cytosolic and receptor), transcription factors, epigenetic writers (e.g., methyltransferases, acetyltransferases,) epigenetic readers, and epigenetic erasers (e.g., demethylases, deacetylases). Examples of methyltransferases include those described in Nature Structural & Molecular Biology volume 26, pages 880-889 (2019), which is incorporated herein by reference in its entirety. Examples of histone demethylases include those described in Nature Reviews Molecular Cell Biology volume 13, pages297-311 (2012), which is incorporated herein by reference in its entirety. More specifically, suitable examples of such proteins include hormone receptor, androgen receptor (AR), estrogen receptor (ER), estrogen-related receptor alpha (ERRα), KRAS, BRD4 (bromodomain and extraterminal (BET) domain epigenetic reader protein BRD4), BRD2, BRD3, anaplastic lymphoma kinase (ALK), BCL2, BCL6, BCR-ABL, BRD9, BRD7, BTK, CDK4/6, cyclin-dependent kinase 8 (CDK8), cyclin-dependent kinase 9 (CDK9), casein kinase 2 (CK2), c-Met, dihydroorotate dehydrogenase (DHODH), epidermal growth factor receptor (EGFR), human epidermal growth factor receptor 2 (HER2), eukaryotic translation initiation factor 4E (eIF4E), ERK1, ERK2, focal adhesion kinase (FAK), FMS-like tyrosine kinase 3 (FLT3), myeloid cell leukemia 1 (MCL1), murine double minute 2 (MDM2), poly(ADP-ribose) polymerase (PARPs, such as PARP1), transforming acidic coiled-coil containing protein 3 (TACC3), pirin, phosphoinositide 3-kinases (P13Ks), polycomb repressive complex 2 (PRC2), serine-threonine kinase (RIPK2), rpn13, serum/glucocorticoid-inducible protein kinase (SGK), smad3, STAT protein (STAT1, STAT2, STAT3, STAT4, STAT5A, STAT5B, or STAT6), TANK-binding kinase 1 (TBK1), TRIM24, the hepatitis C virus (HCV) NS3 protein, interleukin-1 receptor-associated kinase 4 (IRAK4), P300/CBP-associated factor (PCAF), cellular retinoic acid-binding protein (CRABP-I, -II), anaplastic lymphoma kinase (ALK), mitogen-activated protein kinase 14 (MAPK14, p38-α), mitogen-activated protein kinase 13 (MAPK 13, also known as stress-activated protein kinase 4 (SAPK4), or p38-δ), sirtuin, sirtuin2 (SIRT2), P300/CBP associating factor (PCAF), histone deacetylase (e.g., HDAC1, HDAC2, HDAC3, HDAC4, HDAC5, HDAC6,m HDAC7, HDAC8, HDAC9, HDAC10, or HDAC11), cytosolic aminoacyl tRNA synthetase, mitochondrial aminoacyl tRNA synthetase, PD-L1, CD47, cytokine (e.g., IL-2, IL-7, IL-12, IL-15, IL-10, IL-21, or INF-alfa), chemokine (e.g., CCL2, CCL3, or CCL5), and an immunosuppressive antigen (e.g., PD-1, CTLA-4, CD20, Lag-3 or Tim-3). In some embodiments, the protein is implicated in the pathology of a neurodegenerative disease or condition. Suitable examples of such proteins include alpha-synuclein, transthyretin, tau protein, and amyloid-β peptide.

### Selected Definitions

As used herein, the term “about” means “approximately” (e.g., plus or minus approximately 10% of the indicated value).

At various places in the present specification, substituents of compounds of the invention are disclosed in groups or in ranges. It is specifically intended that the invention include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

As used herein, the phrase “optionally substituted” means unsubstituted or substituted. The substituents are independently selected, and substitution may be at any chemically accessible position. As used herein, the term “substituted” means that a hydrogen atom is removed and replaced by a substituent. A single divalent substituent, e.g., oxo, can replace two hydrogen atoms. It is to be understood that substitution at a given atom is limited by valency.

Throughout the definitions, the term “Cₙ₋ₘ” indicates a range which includes the endpoints, wherein n and m are integers and indicate the number of carbons. Examples include C₁₋₄, C₁₋₆, and the like.

As used herein, the term “Cₙ₋ₘ alkyl”, employed alone or in combination with other terms, refers to a saturated hydrocarbon group that may be straight-chain or branched, having n to m carbons. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms. The term “alkylene” includes divalent alkyl groups.

As used herein, the term “Cₙ₋ₘ haloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group has 1 to 6, 1 to 4, or 1 to 3 carbon atoms.

As used herein, “Cₙ₋ₘ alkenyl” refers to an alkyl group having one or more double carbon-carbon bonds and having n to m carbons. Example alkenyl groups include, but are not limited to, ethenyl, n-propenyl, isopropenyl, n-butenyl, sec-butenyl, and the like. In some embodiments, the alkenyl moiety contains 2 to 6, 2 to 4, or 2 to 3 carbon atoms.

As used herein, “halo” refers to F, Cl, Br, or I. In some embodiments, a halo is F, Cl, or Br.

As used herein, the term “aryl,” employed alone or in combination with other terms, refers to an aromatic hydrocarbon group, which may be monocyclic or polycyclic (e.g., having 2, 3 or 4 fused rings). The term “Cₙ₋ₘ aryl” refers to an aryl group having from n to m ring carbon atoms. Aryl groups include, e.g., phenyl, naphthyl, anthracenyl, phenanthrenyl, indanyl, indenyl, and the like. In some embodiments, aryl groups have from 6 to 10 carbon atoms. In some embodiments, the aryl group is phenyl or naphtyl.

As used herein, “Cₙ₋ₘ alkynyl” refers to an alkyl group having one or more triple carbon-carbon bonds and having n to m carbons. Example alkynyl groups include, but are not limited to, ethynyl, propyn-1-yl, propyn-2-yl, and the like. In some embodiments, the alkynyl moiety contains 2 to 6, 2 to 4, or 2 to 3 carbon atoms.

As used herein, the term “Cₙ₋ₘ alkylene”, employed alone or in combination with other terms, refers to a divalent alkyl linking group having n to m carbons. Examples of alkylene groups include, but are not limited to, ethan-1,1-diyl, ethan-1,2-diyl, propan-1,1, -diyl, propan-1,3-diyl, propan-1,2-diyl, butan-1,4-diyl, butan-1,3-diyl, butan-1,2-diyl, 2-methyl-propan-1,3-diyl, and the like. In some embodiments, the alkylene moiety contains 2 to 6, 2 to 4, 2 to 3, 1 to 6, 1 to 4, or 1 to 2 carbon atoms.

As used herein, the term “Cₙ₋ₘ alkoxy”, employed alone or in combination with other terms, refers to a group of formula-O-alkyl, wherein the alkyl group has n to m carbons. Example alkoxy groups include, but are not limited to, methoxy, ethoxy, propoxy (e.g., n-propoxy and isopropoxy), butoxy (e.g., n-butoxy and tert-butoxy), and the like. In some embodiments, the alkyl group has 1 to 6, 1 to 4, or 1 to 3 carbon atoms.

As used herein, “Cₙ₋ₘ haloalkoxy” refers to a group of formula-O-haloalkyl having n to m carbon atoms. An example haloalkoxy group is OCF₃. In some embodiments, the haloalkoxy group is fluorinated only. In some embodiments, the alkyl group has 1 to 6, 1 to 4, or 1 to 3 carbon atoms.

As used herein, the term “amino” refers to a group of formula —NH₂.

As used herein, the term “Cₙ₋ₘ alkylamino” refers to a group of formula-NH (alkyl), wherein the alkyl group has n to m carbon atoms. In some embodiments, the alkyl group has 1 to 6, 1 to 4, or 1 to 3 carbon atoms. Examples of alkylamino groups include, but are not limited to, N-methylamino, N-ethylamino, N-propylamino (e.g., N-(n-propyl)amino and N-isopropylamino), N-butylamino (e.g., N-(n-butyl)amino and N-(tert-butyl)amino), and the like.

As used herein, the term “di(Cₙ₋ₘ-alkyl)amino” refers to a group of formula-N(alkyl)₂, wherein the two alkyl groups each has, independently, n to m carbon atoms. In some embodiments, each alkyl group independently has 1 to 6, 1 to 4, or 1 to 3 carbon atoms.

As used herein, “heteroaryl” refers to a monocyclic or polycyclic aromatic heterocycle having at least one heteroatom ring member selected from sulfur, oxygen, and nitrogen. In some embodiments, the heteroaryl ring has 1, 2, 3, or 4 heteroatom ring members independently selected from nitrogen, sulfur and oxygen. In some embodiments, any ring-forming N in a heteroaryl moiety can be an N-oxide. In some embodiments, the heteroaryl is a 5-10 membered monocyclic or bicyclic heteroaryl having 1, 2, 3 or 4 heteroatom ring members independently selected from nitrogen, sulfur and oxygen. In some embodiments, the heteroaryl is a 5-6 monocyclic heteroaryl having 1 or 2 heteroatom ring members independently selected from nitrogen, sulfur and oxygen. In some embodiments, the heteroaryl is a five-membered or six-membered heteroaryl ring. A five-membered heteroaryl ring is a heteroaryl with a ring having five ring atoms wherein one or more (e.g., 1, 2, or 3) ring atoms are independently selected from N, O, and S. Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl. A six-membered heteroaryl ring is a heteroaryl with a ring having six ring atoms wherein one or more (e.g., 1, 2, or 3) ring atoms are independently selected from N, O, and S. Exemplary six-membered ring heteroaryls are pyridyl, pyrazinyl, pyrimidinyl, triazinyl and pyridazinyl.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, N═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. In some embodiments, the compound has the (R)-configuration. In some embodiments, the compound has the(S)-configuration.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

As used herein, the term “contacting” refers to the bringing together of indicated moieties in an in vitro system or an in vivo system.

### EXPERIMENTS

Compound synthesis: All small molecule reagents are synthesized on 5-25 mg scale to allow for proper characterization and comprehensive testing. All compounds purified to >95% purity and characterized by ¹H-NMR, ¹³C-NMR, and LC/MS. Compound stock solutions will be stored at −80° C., and dry substances stored at ° C.

Photophysical characterization: determine absorbance and emission spectra, quantum yield, and q-value. Excitation and emission kinetics acquired using custom profiling platform, which provides sub-microsecond resolution and is even suited for Sm and Dy complexes (FIG. 7). All measurements performed using at least three technical replicates and include CoraFluor-1 as a reference standard.

Physicochemical characterization: measured logD values and stability towards various buffers and common excipients as previously reported. All measurements performed using at least three technical replicates and include CoraFluor-1 as a reference standard.

Performance benchmark assays: To evaluate CoraFluors in cell-free biochemical assays, a robust benchmarking platform used that uses self-labeling protein tags. Specifically, cloned and expressed an engineered HaloTag-SNAP-tag fusion protein containing a C-terminal His6 tag. The modular design enables the orthogonal labeling of individual domains with small molecule probes (e.g., fluorophores, affinity handles) in defined stoichiometries, resembling ligand binding events and/or protein-protein interactions without potentially confounding effects due to ligand dissociation at high dilutions. anti-IgG nanobody platform reported previously can also be used. This allows to characterize new CoraFluors analogs in the context of larger protein complexes.

Statistics and data analysis. Experiments conducted with at least 3 technical replicates and repeated independently at least twice. Dose-response data fit by linear regression analysis. Biological replicates are not required.

### Experiment 1

Next-generation CoraFluor ligands developed with improved performance and extended applicability, including TR-FRET probes with improved brightness, enhanced cell permeability, extended TR-FRET range, optimized characteristics for imaging applications and compatibility with other lanthanides. Probes developed also useful for regular fluorescence and TRF applications. CoraFluors are very resistant to photobleaching and can be used under steady-state illumination.

### Experiment 2

TR-FRET microarray scanners are not available due to non-existence of the novel and non-obvious microarray approach within the present claims. However, such instruments, which essentially constitute simple TR-fluorescent microscopes that control excitation and delay with inexpensive mechanical components (chopper), will be easier and cheaper to manufacture than traditional laser-scanning readers while enabling higher throughput and continuous readout.

Small-molecule and peptide arrays: commercially available streptavidin, NHS, or maleimide functionalized microtiter plates (e.g., Acro Biosystems, Automate Scientific, Poly-An) can be used to immobilize small molecule ligands that are amine-modified or sulfhydryl-modified, respectively. For these studies, FKBP-12 binders can be employed with differential affinities. BRD4 as well as a set of HDAC inhibitor ligands can be used as non-limiting examples, and many other ligands and proteins can be used.

Stock solutions of the ligands can be prepared with varying ratios of linker-functionalized CoraFluors (e.g., 100:1, 10:1, 1:1, 1:10). This allows to simultaneously functionalize the plate surface and determine the optimal ratio of TR-FRET donor and “bait” molecules. Plates incubated with these stock solutions, washed, incubated with biotin/ethanol amine/mercaptoethanol to block unreacted sites, and washed again. Accordingly, functionalized plates incubated with varying concentrations of fluorescently tagged proteins, including positive and negative controls, and analyzed. The bottom area of a single well of a 96-well plate is ~0.3 cm², and the cross-section of a regular protein is ~20 nm². Complete coverage of the well bottom with a protein monolayer requires ~10¹² proteins, which, if distributed in a well volume of 100 μL, corresponds to a concentration of ~10 nM. CoraFluor limit of detection is <100 fM, offering >4 orders of magnitude dynamic range. This allows to quantify equilibrium binding to fewer than 0.001% of the available sites. Since most plate readers, including the Tecan Spark used for these studies, enable readout focused on the well-bottom, the numbers above represent a conservative estimate, and the detection sensitivity is likely considerably higher. To perform off-rate measurements, the experiment saturated ligand binding at equilibrium conditions, followed by the addition of an unlabeled competitor and continues measurement of the TR-FRET signal.

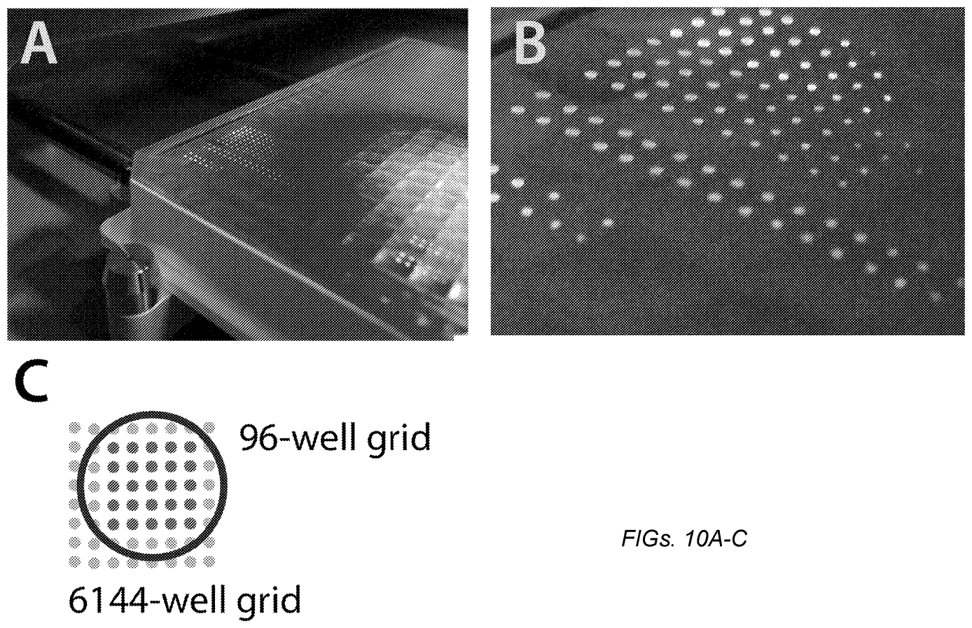

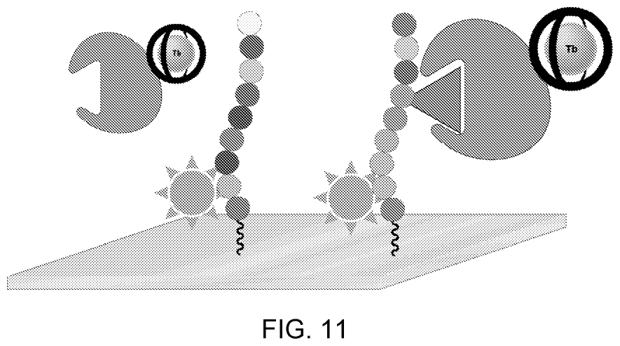

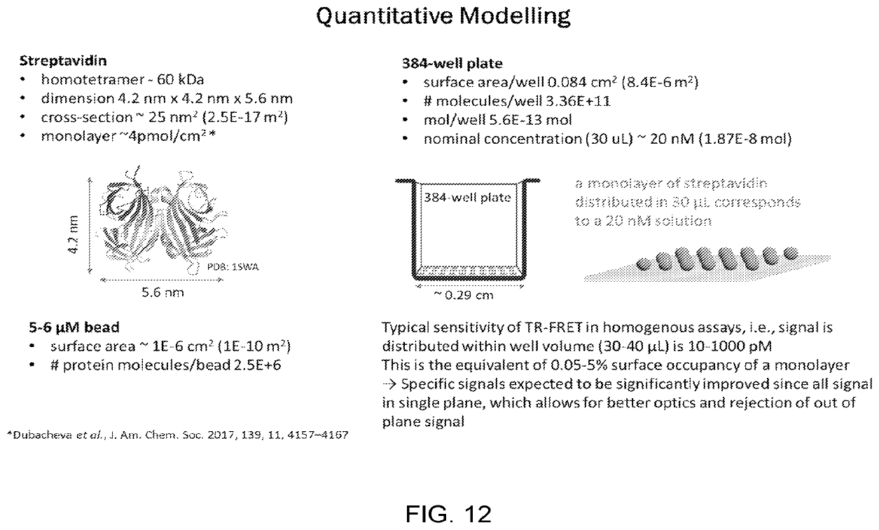

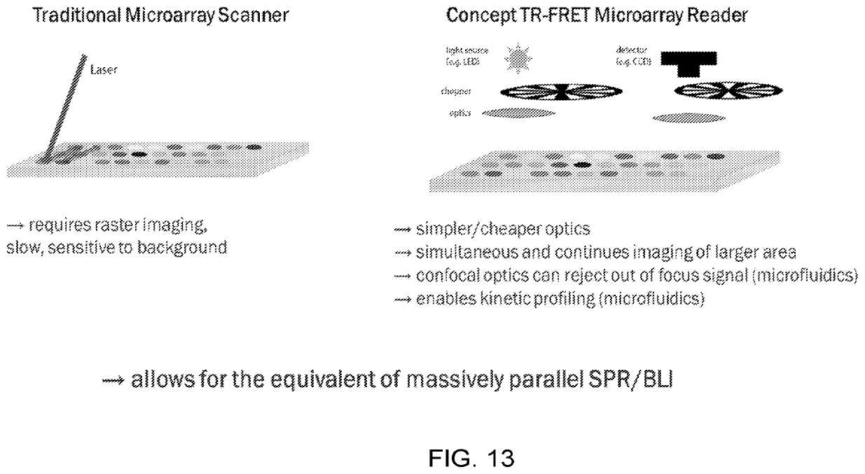

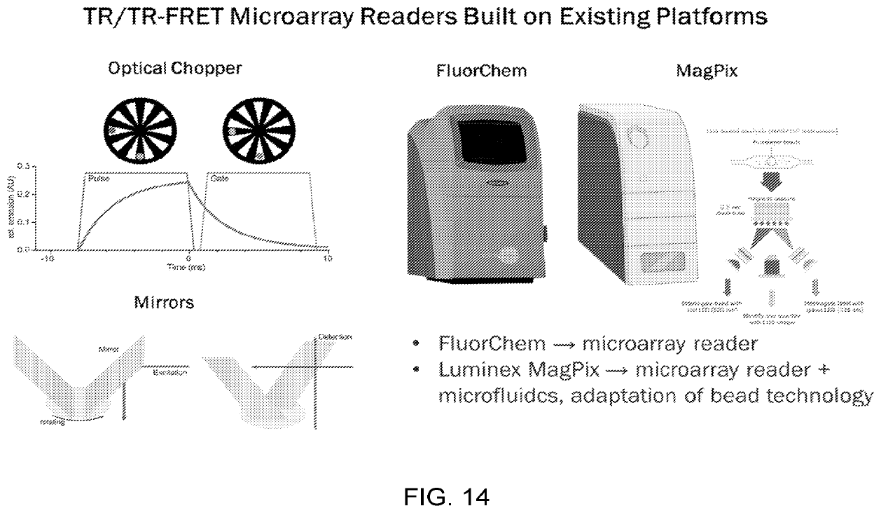

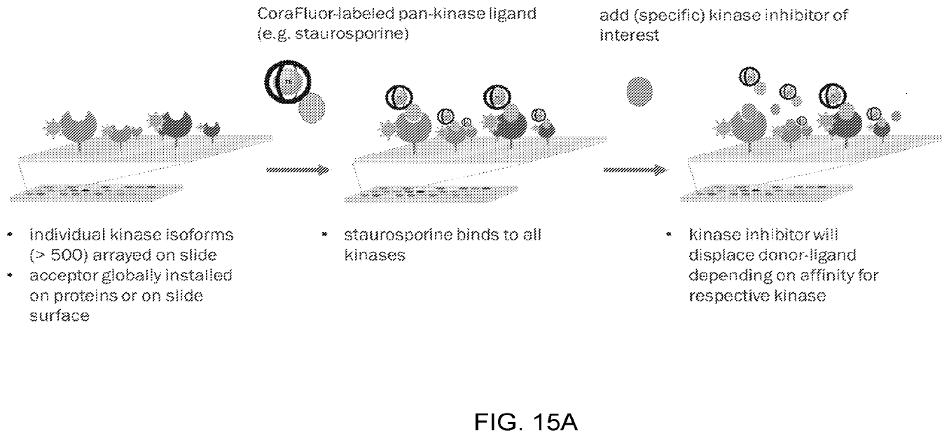

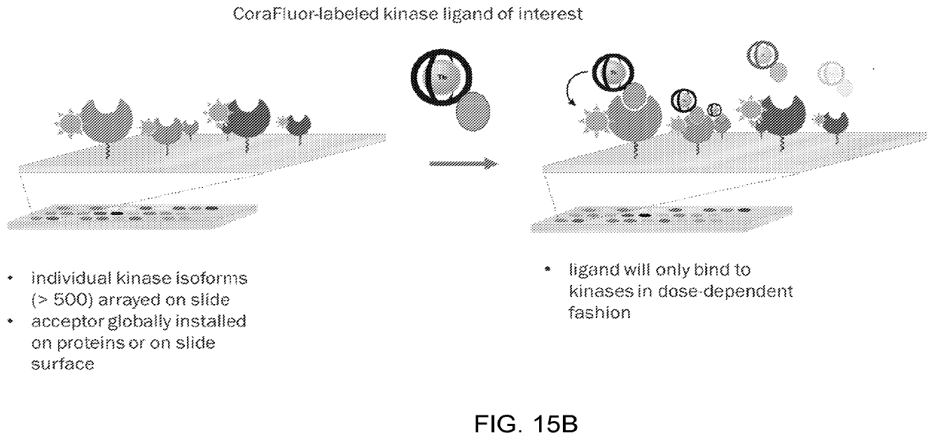

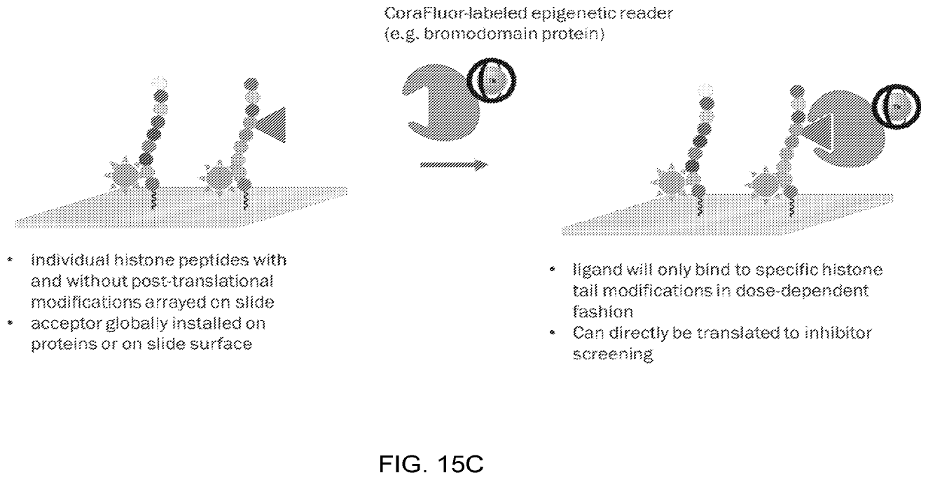

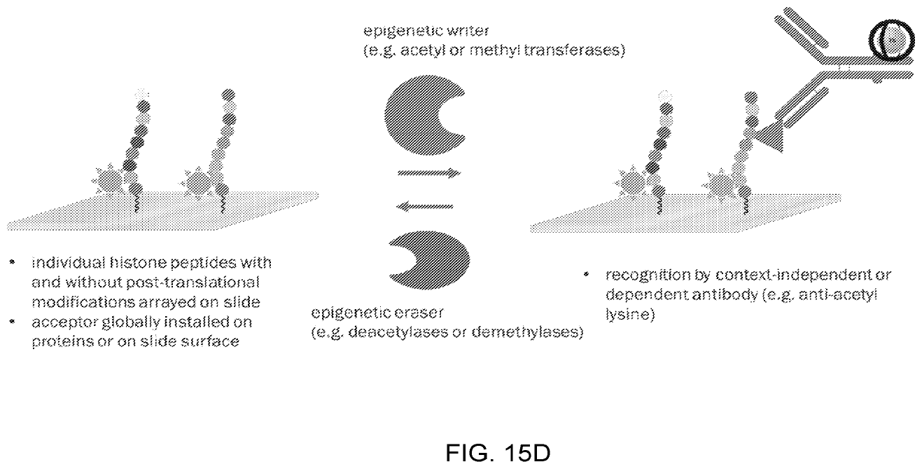

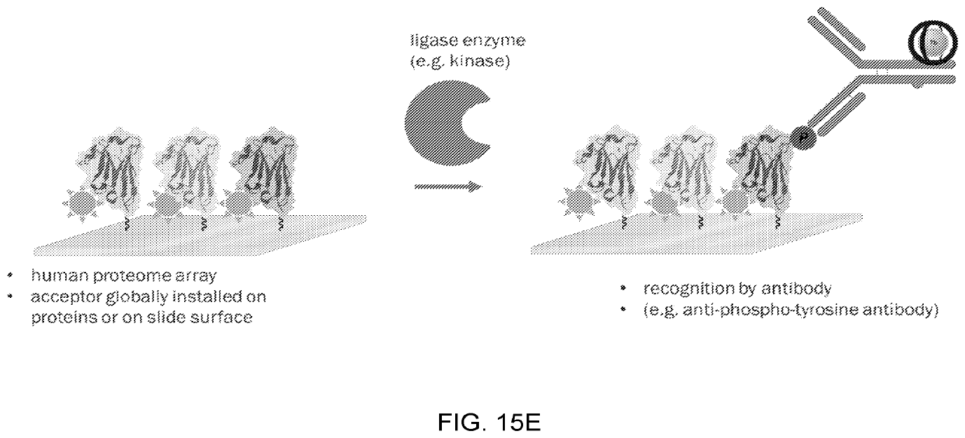

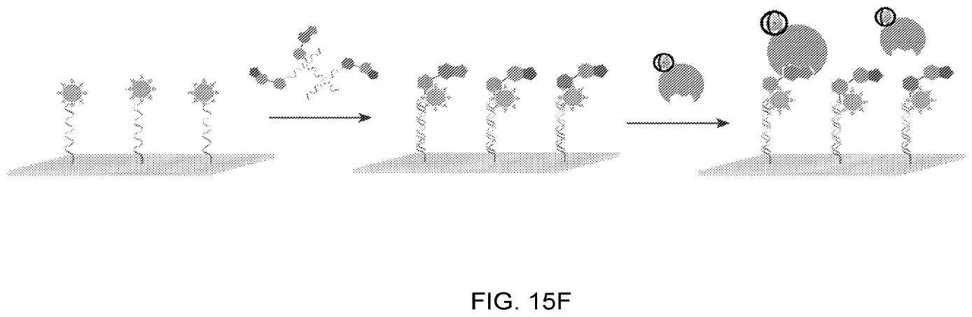

For peptide arrays, commercially available biotinylated histone peptides (Alta Bioscience) can be used, among others, with distinct posttranslational modifications, including lysine methylation, acetylation, and phosphorylation. Streptavidin-coated plates modified with Alexfluor488 (as above) and immobilized with the respective histone peptides. To deposit the peptide stock solutions, HP D300 liquid dispenser can be used (FIG. 10), which can be used to deposit defined nL quantities inside the wells in a grid format, which can be scanned with the plate reader (FIG. 9D). CoraFluor-1-labeled bromodomain proteins (acetyl marks) and chromodomain proteins, and histone sequence-specific antibodies can then be added. The approach can be translated to functionalized glass slides.

### Experiment 3

A TR-FRET microarray systems allow for more sensitive, flexible, and even no-wash compatible microarray setups. As illustrated in the figures energy transfer from surface bound CoraFluors (or other TR-FRET donors) to a nearby bound acceptor-labeled protein (or other biomolecule or molecule of interest) provides specific TR-FRET signal. The TR-FRET donor can be installed via doping of the microarray surface, or through other means.

Small molecule microarrays represent an extremely promising way to profile large numbers of immobilized molecules against protein targets in high-throughput. The major drawbacks of many of these technologies is the requirement for extensive wash-steps to remove nonspecific signal, which limits application to high-affinity interactions. Current small molecule microarray technology does not take advantage of the sensitivity that TR-FRET offers. By combining the sensitivity of time-resolved fluorescence with the specificity of FRET, wash-free microarray technologies are developed that allow: Applicability to lower affinity ligands. Greatly simplify assay workflow. Increase sensitivity orders of magnitude. Enable semi-quantitative characterization of binding affinities and kinetics.

### Procedure—Labeling Surface of SA-Coated 384-Well Plates

Plates—Perkin Elmer (delfia-sa-white-plate-384-wel). Reaction buffer-100 mM sodium carbonate, pH 8.5+0.05% TWEEN-20. Assay Buffer-25 mM HEPES, 150 mM NaCl, 0.5 mg/mL BSA, 0.005% TWEEN-20, pH 7.5. AF488-Tfp is available from Fisher (A37570).

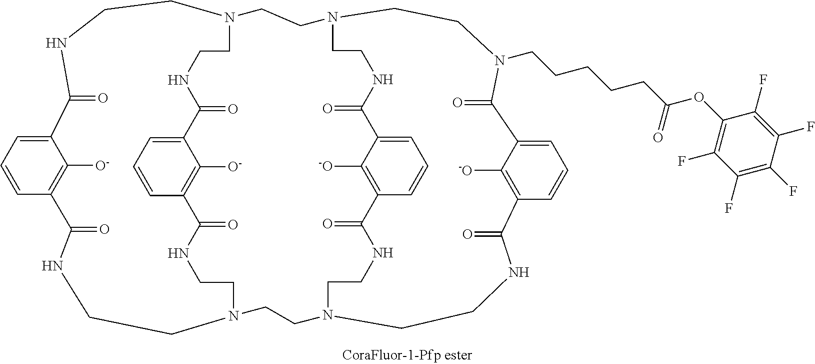

Covalently label SA surface with 25 μM CoraFluor-1-Pfp ester or 25 μM AF488-Tfp ester in reaction buffer for 30 min at rt. Wash 5× with assay buffer until fluorescence signal stabilizes on Tecan. Block half wells with 100 μM biotin 20 min rt. Add NCP357 or NCP358-Tb to CoraFluor-1 or AF488-labeled wells, respectively and read TR-FRET signal relative to wells pre-blocked with biotin.

Labeled wells with 25 μM CoraFluor-1-Pfp ester in reaction buffer. Labeled wells with 25 μM AF488-Tfp ester in reaction buffer 30 min rt. Aspirated solution and washed with assay buffer (80 μL/well) and read 490 nm TR fluorescence intensity and 520 nm FLINT on Tecan. Repeated 4×until signal stabilized, indicating clear labeling of the surface. Incubated some wells with 1% DMSO in assay buffer 20 min rt. Incubated some wells with 100 μM biotin in assay buffer 20 min rt.

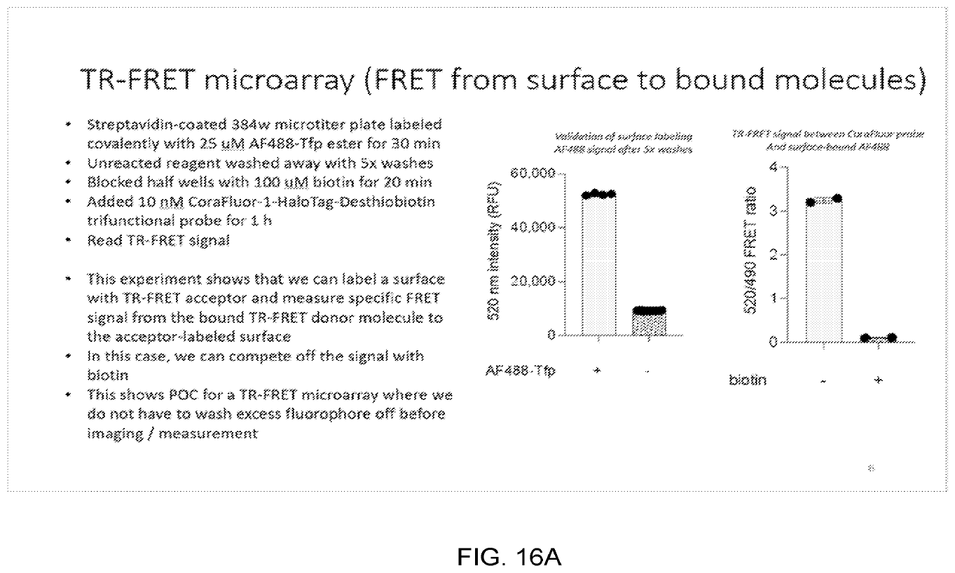

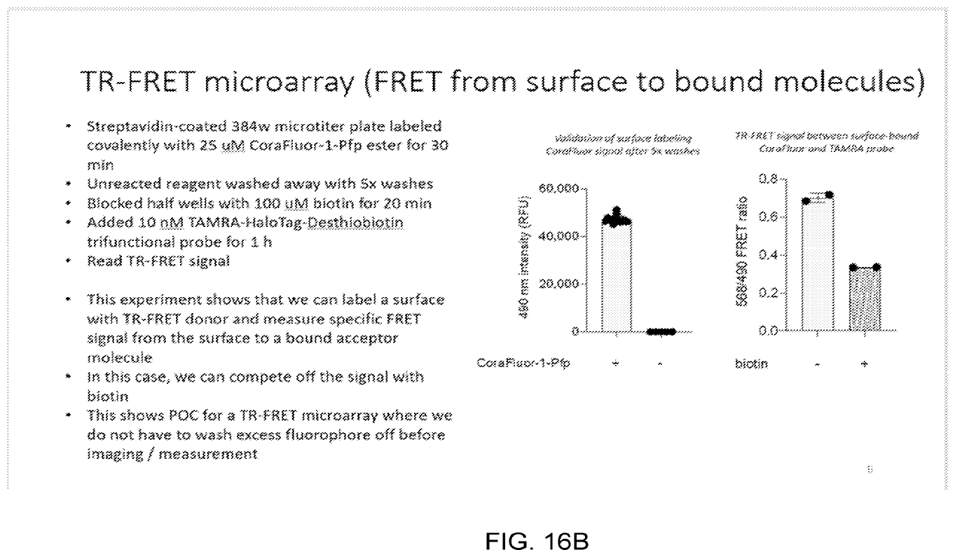

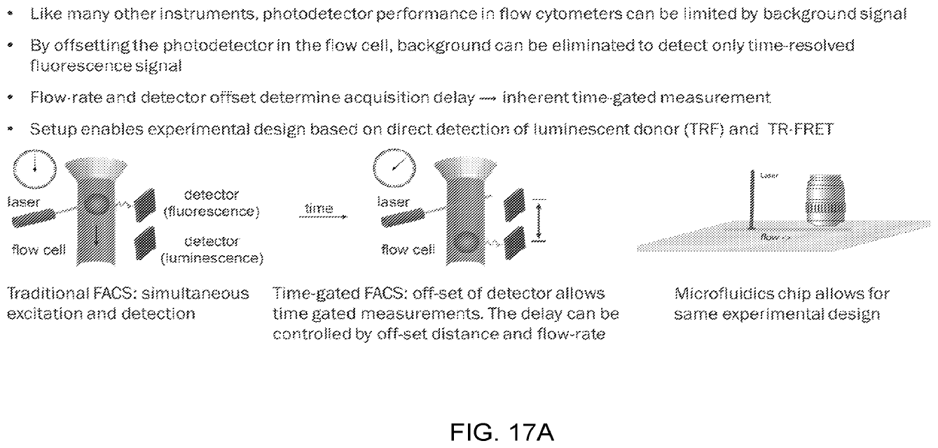

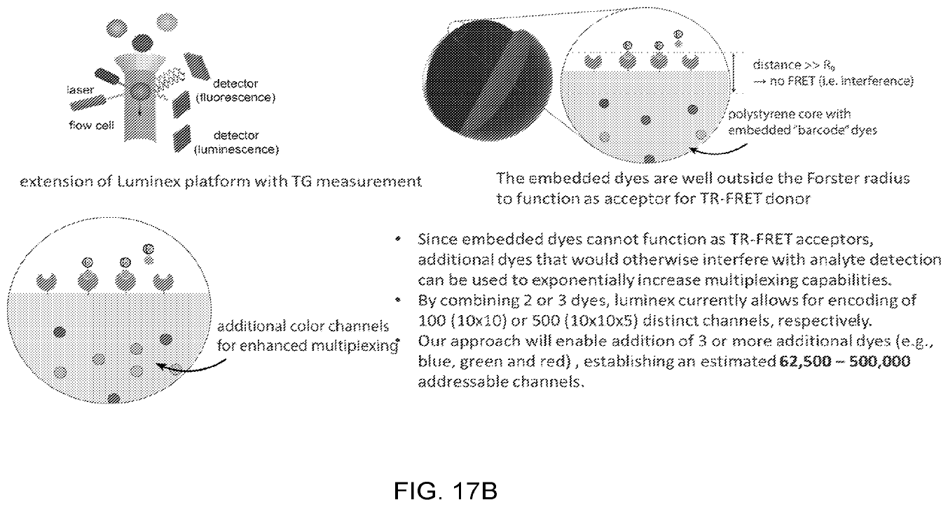

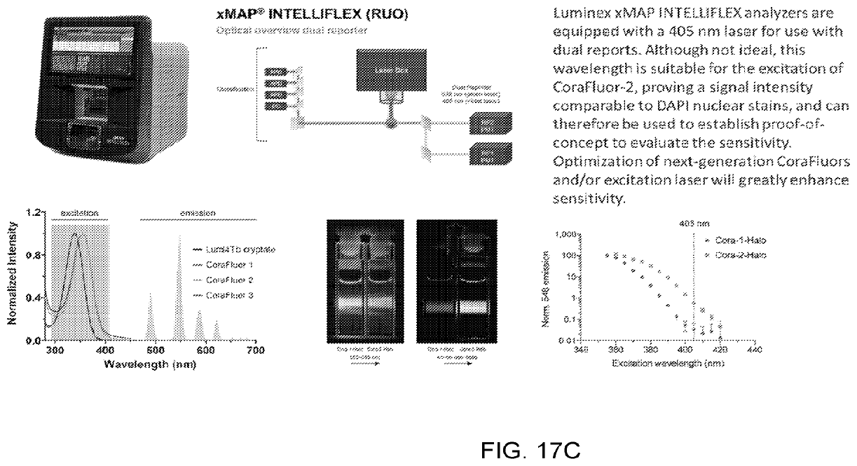

Results are shown in FIGS. 16A and 16B. For 10 nM conditions (CoraFluor-labeled surface), see clear specific TR-FRET signal at t=5 min and t=1 h. For 10 nM conditions (AF488-labeled surface), see clear specific TR-FRET signal at t=5 min and t=1 h. For 100 nM conditions (CoraFluor-labeled surface), see clear specific TR-FRET signal at t=5 min. For 1000 nM conditions (CoraFluor-labeled surface), did not see much specific TR-FRET signal, even when shortening delay and acquisition. These results clearly demonstrate proof of concept for a TR-FRET microarray where either donor surface bound and FRET from the donor-surface to bound acceptor, or acceptor surface bound and FRET occurs from bound donor to the acceptor-labeled surface. Both systems (surface donor and surface acceptor) result in superior performance and allow no-wash conditions in TR-FRET microarray systems.

### OTHER EMBODIMENTS

It is to be understood that while the present application has been described in conjunction with the detailed description thereof, the foregoing description is intended to illustrate and not limit the scope of the present application, which is defined by the scope of the appended claims. Other aspects, advantages, and modifications are within the scope of the following claims.

1. A method for detecting an interaction of a target molecule and a ligand molecule, the method comprising:
(i) treating a surface modified with a target molecule, wherein either the surface or the target molecule is further modified with a FRET acceptor moiety, with a ligand molecule, wherein the ligand molecule is modified with a FRET donor moiety; and
(ii) exciting the surface with a light of a wavelength capable of being absorbed by the FRET donor moiety, waiting an amount of time sufficient for energy transfer from the FRET donor moiety to the FRET acceptor moiety, and detecting a light of a wavelength emitted by the FRET acceptor moiety by a fluorescence imaging technique.

2. The method of claim 1, wherein the target molecule is selected from small molecule, protein, peptide, nucleic acid, lipid, and carbohydrate.

3. The method of claim 1, wherein the ligand molecule is selected from small molecule, protein, peptide, nucleic acid, lipid, and carbohydrate.

4. The method of claim 1, wherein the target molecule is a small molecule and the ligand molecule is a protein.

5. The method of claim 1, wherein the target molecule and the ligand molecule are proteins.

6. The method of claim 1, wherein the target molecule is a peptide and the ligand molecule is a protein.

7. The method of claim 1, wherein the ligand molecule is a protein selected from an enzyme, a cell-surface receptor, nuclear hormone receptor, a transporter, a G-protein coupled receptor, a CD marker, a voltage-gated ion channel, a nuclear factor, a nuclear receptor, a protein-protein or protein-peptide interaction domain, scaffolding protein, structural protein, transcription factor, chaperone, and assembly/disassembly factor.

8. The method of claim 1, wherein the target molecule and the ligand molecule are both nucleic acids.

9. The method of claim 1, wherein the surface is a glass or a silicon slide.

10. The method of claim 1, wherein the FRET acceptor moiety is a fluorophore.

11. The method of claim 10, wherein the fluorophore is selected from fluorescein, AF488, hydroxycoumarin blue, methoxycoumarin blue, alexa fluor blue, aminocoumarin blue, Cy2 green (dark), FAM green (dark), alexa fluor 488 green (light), fluorescein FITC green (light), alexa fluor 430 green (light), Alexa fluor 532 green (light), HEX green (light), Cy3 yellow, TRITC yellow, Alexa fluor 546 yellow, Alexa fluor 555 3 yellow, R-phycoerythrin (PE) 480; yellow, Rhodamine Red-X orange, Tamara red, Cy3.5 581 red, Rox red, Alexa fluor 568 red, Red 613 red, Texas Red red, Alexa fluor 594 red, Alexa fluor 633 red, Allophycocyanin red, Alexa fluor 633 red, Cy5 red, Alexa fluor 660 red, Cy5.5 red, TruRed red, Alexa fluor 680 red, and Cy7 red.

12. The method of claim 1, wherein said detecting of fluorescence is carried out using fluorescent microscopy, fluorescent imaging probe, or fluorescent spectroscopy.

13. The method of claim 1, wherein the method comprises evaluating an interaction between the target molecule and the ligand molecule.

14. The method of claim 1, wherein detecting the light of the wavelength emitted by the FRET acceptor moiety is a detection of a fluorescence intensity from the FRET acceptor moiety and is indicative of quality and/or quantity of the interaction between the target molecule and the ligand molecule.

15. The method of claim 1, wherein the FRET donor moiety comprises a complex of a lanthanide metal with a moiety of formula (i): 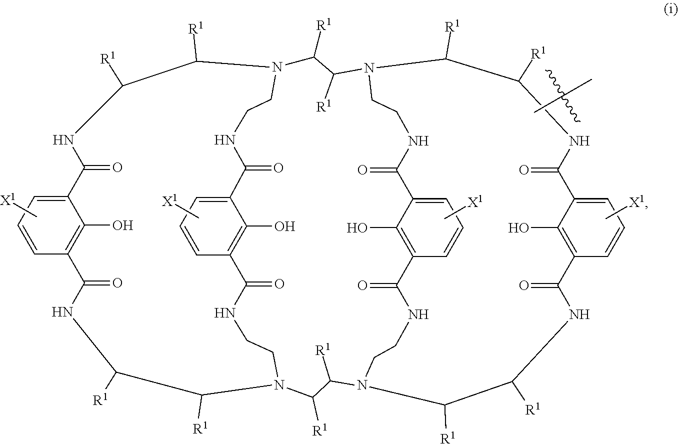
wherein:
each X¹ is independently selected from halo, NO₂, CN, N₃, C₁₋₆ alkyl, C₁₋₆ alkoxy, C₂₋₆ alkenyl, C₂₋₆ alkynyl, C₆₋₁₀ aryl, and 5-14 membered heteroaryl, wherein said C₁₋₆ alkyl, C₂₋₆ alkenyl, C₂₋₆ alkynyl, C₆₋₁₀ aryl, and 5-14 membered heteroaryl are each optionally substituted with 1, 2, or 3 substituents independently selected from halo, OH, SH, NH₂, C₁₋₃ alkylamino, di(C₁₋₃ alkyl)amino, NO₂, CN, C(O)OH, C₁₋₃ alkoxy, C₁₋₃ haloalkoxy, and N₃; and
each R¹ is independently selected from H, C₁₋₆ alkyl, C₂₋₆ alkenyl, and C₂₋₆ alkynyl, each of which is optionally substituted with 1, 2, or 3 substituents independently selected from halo, OH, SH, NH₂, C₁₋₃ alkylamino, di(C₁₋₃ alkyl)amino, NO₂, CN, C(O)OH, C₁₋₃ alkoxy, C₁₋₃ haloalkoxy, and N₃.

16. The method of claim 15, wherein the moiety of formula (i) has any one of the following formula:

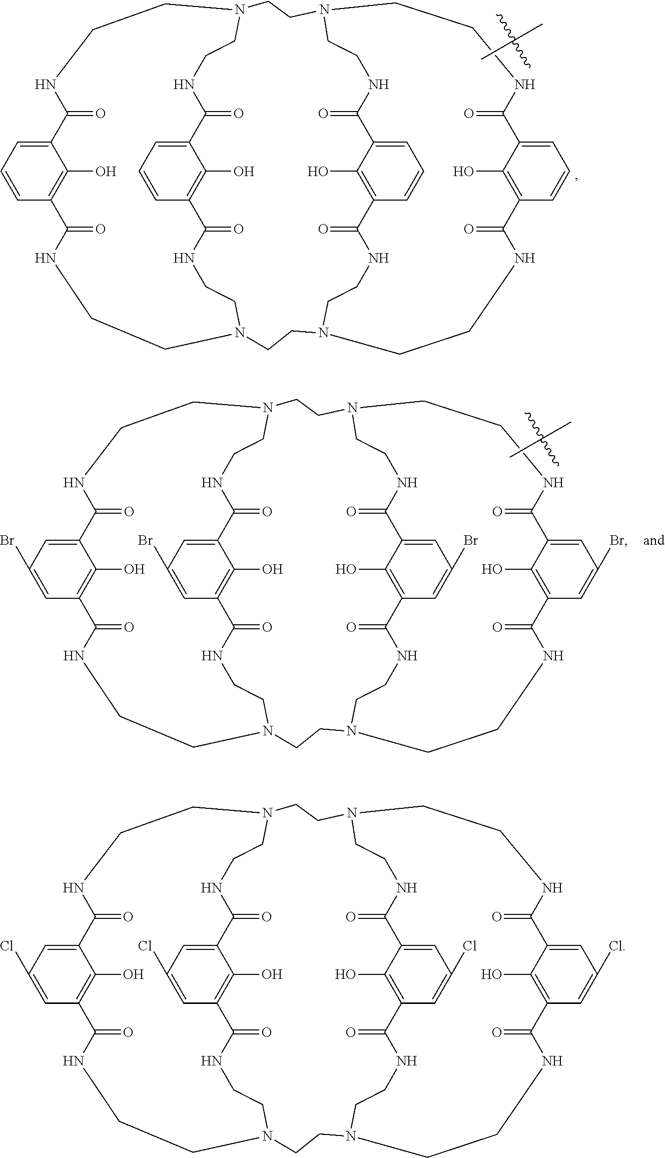

17. The method of claim 16, wherein the lanthanide metal is selected from Tb (terbium), Eu (europium), Sm (samarium), and Dy (dysprosium).

18. The method of claim 17, wherein the lanthanide metal is Tb³⁺.

19. A method for detecting an interaction of a target molecule and a ligand molecule, the method comprising:
(i) treating a surface modified with a target molecule, wherein either the surface or the target molecule is further modified with a FRET donor moiety,
with a ligand molecule, wherein the ligand molecule is modified a FRET acceptor moiety; and
(ii) exciting the surface with a light of a wavelength capable of being absorbed by the FRET donor moiety, waiting an amount of time sufficient for energy transfer from the FRET donor moiety to the FRET acceptor moiety, and detecting a light of a wavelength emitted by the FRET acceptor moiety by a fluorescence imaging technique.

20. A method of identifying a compound that modulates a protein of interest, the method comprising:
(i) treating a surface modified with a target molecule, wherein either the surface or the target molecule is further modified with a FRET acceptor moiety,
with a protein of interest, wherein the protein of interest is modified with a FRET donor moiety;
(ii) exciting the surface with a light of a wavelength capable of being absorbed by the FRET donor moiety, waiting an amount of time sufficient for energy transfer from the FRET donor moiety to the FRET acceptor moiety, and detecting a light of a wavelength emitted by the FRET acceptor moiety by a fluorescence imaging technique;
(iii) treating the surface with a test compound;
(iv) after (iii), detecting a light of a wavelength emitted by the FRET acceptor moiety by the fluorescence imaging technique; and
(v) determining whether an intensity of fluorescence detected from the FRET acceptor moiety in step (iv) is decreased compared to an intensity of fluorescence detected from the FRET acceptor moiety in step (ii),
wherein said decrease in fluorescence intensity in an indication that the test compound is a modulator the protein of interest.
